(19) United States
(12) Patent Application Publication
(10) Pub. No.: US 2003/0225127 A1
(43) Pub. Date: Dec. 4, 2003

(54) Heterocyclic sulfonamide derivatives

(76) Inventors: David Michael Bender, Indinapolis, IN (US); Scott Louis Forman, Indianapolis, IN (US); Winton Dennis Jones, Carmel, IN (US); Daryl Lynn Smith, Fishers, IN (US); Hamideh Zarrinmayeh, Carmel, IN (US); Dennis Michael Zimmerman, Zionsville, IN (US)

Correspondence Address: ELI LILLY AND COMPANY, PATENT DIVISION, P.O. BOX 6288, INDIANAPOLIS, IN, 46206-6288, US

(21) Appl. No.: 10/343,186

(22) PCT Filed: Jul. 27, 2001
(86) PCT No.: PCT/US01/21121

(51) Int. Cl. A61K 31/4706; A61K 31/428; A61K 31/423; A61K 31/4184; A61K 31/405

(52) U.S. Cl. 514/312; 514/367; 514/375; 514/394; 514/418; 546/158; 548/165; 548/221; 548/306.4; 548/484

(57) ABSTRACT

The present invention provides certain heterocyclic sulfonamide derivatives of formula (I): useful for potentiating glutamate receptor function in a patient and therefore, useful for treating a wide variety of conditions, such as psychiatric and neurological disorders.

### BACKGROUND OF THE INVENTION

In the mammalian central nervous system (CNS), the transmission of nerve impulses is controlled by the interaction between a neurotransmitter, that is released by a sending neuron, and a surface receptor on a receiving neuron, which causes excitation of this receiving neuron. L-Glutamate, which is the most abundant neurotransmitter in the CNS, mediates the major excitatory pathway in mammals, and is referred to as an excitatory amino acid (EAA). The receptors that respond to glutamate are called excitatory amino acid receptors (EAA receptors). See Watkins & Evans, Ann. Rev. Pharmacol. Toxicol., 21, 165 (1981); Monaghan, Bridges, and Cotman, Ann. Rev. Pharmacol. Toxicol., 29, 365 (1989); Watkins, Krogsgaard-Larsen, and Honore, Trans. Pharm. Sci., 11, 25 (1990). The excitatory amino acids are of great physiological importance, playing a role in a variety of physiological processes, such as long-term potentiation (learning and memory), the development of synaptic plasticity, motor control, respiration, cardiovascular regulation, and sensory perception.

Excitatory amino acid receptors are classified into two general types. Receptors that are directly coupled to the opening of cation channels in the cell membrane of the neurons are termed “ionotropic”. This type of receptor has been subdivided into at least three subtypes, which are defined by the depolarizing actions of the selective agonists N-methyl-D-aspartate (NMDA), alpha-amino-3-hydroxy-5-methylisoxazole-4-propionic acid (AMPA), and kainic acid (KA). The second general type of receptor is the G-protein or second messenger-linked “metabotropic” excitatory amino acid receptor. This second type is coupled to multiple second messenger systems that lead to enhanced phosphoinositide hydrolysis, activation of phospholipase D, increases or decreases in c-AMP formation, and changes in ion channel function. Schoepp and Conn, Trends in Pharmacol. Sci., 14, 13 (1993). Both types of receptors appear not only to mediate normal synaptic transmission along excitatory pathways, but also participate in the modification of synaptic connections during development and throughout life. Schoepp, Bockaert, and Sladeczek, Trends in Pharmacol. Sci., 11, 508 (1990); McDonald and Johnson, Brain Research Reviews, 15, 41 (1990).

AMPA receptors are assembled from four protein sub-units known as GluR1 to GluR4, while kainic acid receptors are assembled from the sub-units GluR5 to GluR7, and KA-1 and KA-2. Wong and Mayer, Molecular Pharmacology 44: 505-510, 1993. It is not yet known how these sub-units are combined in the natural state. However, the structures of certain human variants of each sub-unit have been elucidated, and cell lines expressing individual sub-unit variants have been cloned and incorporated into test systems designed to identify compounds which bind to or interact with them, and hence which may modulate their function. Thus, European patent application, publication number EP-A2-0574257 discloses the human sub-unit variants GluR1B, GluR2B, GluR3A and GluR3B. European patent application, publication number EP-A1-0583917 discloses the human sub-unit variant GluR4B.

One distinctive property of AMPA and kainic acid receptors is their rapid deactivation and desensitization to glutamate. Yamada and Tang, The Journal of Neuroscience, September 1993, 13(9): 3904-3915 and Kathryn M. Partin, J. Neuroscience, Nov. 1, 1996, 16(21): 6634-6647.

It is known that the rapid desensitization and deactivation of AMPA and/or kainic acid receptors to glutamate may be inhibited using certain compounds. This action of these compounds is often referred to in the alternative as “potentiation” of the receptors. One such compound, which selectively potentiates AMPA receptor function, is cyclothiazide. Partin et al., Neuron. Vol. 11, 1069-1082, 1993.

International Patent Application Publication WO 98/33496 published Aug. 6, 1998 discloses certain sulfonamide derivatives which are useful, for example, for treating psychiatric and neurological disorders, for example cognitive disorders; neuro-degenerative disorders such as Alzheimer's disease; age-related dementias; age-induced memory impairment; movement disorders such as tardive dyskinesia, Huntington's chorea, myoclonus, and Parkinson's disease; reversal of drug-induced states (such as cocaine, amphetamines, alcohol-induced states); depression; attention deficit disorder; attention deficit hyperactivity disorder; psychosis; cognitive deficits associated with psychosis, and drug-induced psychosis.

### SUMMARY OF THE INVENTION

The present invention provides compounds of formula I:

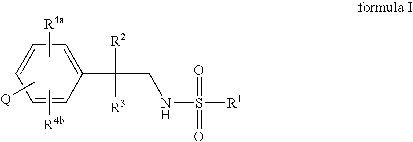

wherein

R¹ represents (1-6C)alkyl, (2-6C)alkenyl, or NR⁷R⁸;

R² and R³ each independently represent hydrogen, (1-4C)alkyl, or —OR⁹;

R^{4a} and R^{4b} each independently represent hydrogen, (1-4C) alkyl, (1-4C)alkoxy, I, Br, Cl, or F; and

Q is selected from the following:

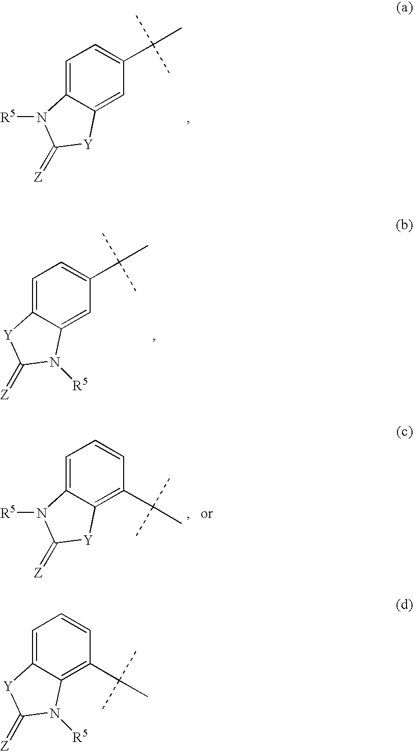

wherein

R⁵ represents hydrogen or (1-6C)alkyl;

Y represents CH₂CH₂, CR¹⁰R¹¹, NR⁶, S, or O;

Z represents O, S, or NH;

R⁶ represents hydrogen or (1-6C)alkyl;

R⁷ and R⁸ each independently represent hydrogen or (1-4C)alkyl;

R⁹ represents hydrogen or (1-4C)alkyl; and

R¹⁰ and R¹¹ each independently represent hydrogen or (1-4C)alkyl;

or a pharmaceutically acceptable salt thereof.

The present invention further provides a method of potentiating glutamate receptor function in a patient, which comprises administering to said patient an effective amount of a compound of formula I.

The present invention provides a method of treating cognitive disorders in a patient, which comprises administering to said patient an effective amount of a compound of formula I.

In addition, the present invention further provides a method of treating cognitive deficits associated with psychosis in a patient, which comprises administering to said patient an effective amount of a compound of formula I.

According to another aspect, the present invention provides the use of a compound of formula I, or a pharmaceutically acceptable salt thereof for the manufacture of a medicament for potentiating glutamate receptor function.

In addition, the present invention provides the use of a compound of formula I or a pharmaceutically acceptable salt thereof for potentiating glutamate receptor function.

The invention further provides pharmaceutical compositions comprising, a compound of formula I and a pharmaceutically acceptable diluent or carrier.

This invention also encompasses novel intermediates, and processes for the synthesis of the compounds of formula I.

### DETAILED DESCRIPTION OF THE INVENTION

In this specification, the term “potentiating glutamate receptor function” refers to any increased responsiveness of glutamate receptors, for example AMPA receptors, to glutamate or an agonist, and includes but is not limited to inhibition of rapid desensitization or deactivation of AMPA receptors to glutamate.

As used herein the term “AMPA receptor potentiator” refers to a compound which inhibits the rapid desensitization or deactivation of AMPA receptors to glutamate.

A wide variety of conditions may be treated or prevented by compounds of formula I and their pharmaceutically acceptable salts through their action as potentiators of glutamate receptor function. Such conditions include those associated with glutamate hypofunction, such as psychiatric and neurological disorders, for example cognitive disorders and neuro-degenerative disorders such as Alzheimer's disease; age-related dementias; age-induced memory impairment; cognitive deficits due to autism, Down's syndrome and other central nervous system disorders with childhood onset, cognitive deficits post electroconvulsive therapy, movement disorders such as tardive dyskinesia, Hungtington's chorea, myoclonus, dystonia, spasticity, and Parkinson's disease; reversal of drug-induced states (such as cocaine, amphetamines, alcohol-induced states); depression; attention deficit disorder; attention deficit hyperactivity disorder; psychosis; cognitive deficits associated with psychosis, drug-induced psychosis, stroke, and sexual dysfunction. Compounds of formula I may also be useful for improving memory (both short term and long term) and learning ability. The present invention provides the use of compounds of formula I for the treatment of each of these conditions.

The present invention includes the pharmaceutically acceptable salts of the compounds defined by formula I. A compound of this invention can possess a sufficiently acidic group, a sufficiently basic group, or both functional groups, and accordingly react with any of a number of organic and inorganic bases, and inorganic and organic acids, to form a pharmaceutically acceptable salt. The term “pharmaceutically acceptable salt” as used herein, refers to salts of the compounds of the above formula which are substantially non-toxic to living organisms. Typical pharmaceutically acceptable salts include those salts prepared by reaction of the compounds of the present invention with a pharmaceutically acceptable mineral or organic acid or an organic or inorganic base. Such salts are known as acid addition and base addition salts. Such salts include the pharmaceutically acceptable salts listed in Journal of Pharmaceutical Science, 66, 2-19 (1977) which are known to the skilled artisan. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, iodide, acetate, propionate, decanoate, caprate, caprylate, acrylate, ascorbate, formate, hydrochloride, dihydrochloride, hydrobromide, isobutyrate, caproate, heptanoate, propiolate, propionate, phenylpropionate, salicylate, oxalate, malonate, succinate, suberate, sebacate, fumarate, malate, maleate, hydroxymaleate, mandelate, nicotinate, isonicotinate, cinnamate, hippurate, nitrate, phthalate, teraphthalate, butyne-1,4-dioate, butyne-1,4-dicarboxylate, hexyne-1,4-dicarboxylate, hexyne-1,6-dioate, benzoate, chlorobenzoate, methylbenzoate, hydroxybenzoate, methoxybenzoate, dinitrobenzoate, o-acetoxybenzoate, naphthalene-2-benzoate, phthalate, p-toluenesulfonate, p-bromobenzenesulfonate, p-chlorobenzenesulfonate, xylenesulfonate, phenylacetate, trifluoroacetate, phenylpropionate, phenylbutyrate, citrate, lactate, α-hydroxybutyrate, glycolate, tartrate, benzenesulfonate, methanesulfonate, ethanesulfonate, propanesulfonate, hydroxyethanesulfonate, 1-naphthalenesulfonate, 2-napththalenesulfonate, 1,5-naphthalenedisulfonate, mandelate, tartarate, and the like. Preferred pharmaceutically acceptable acid addition salts are those formed with mineral acids such as hydrochloric acid and hydrobromic acid, and those formed with organic acids such as maleic acid, oxalic acid and methanesulfonic acid.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred.

It should be recognized that the particular counterion forming a part of any salt of this invention is usually not of a critical nature, so long as the salt as a whole is pharmacologically acceptable and as long as the counterion does not contribute undesired qualities to the salt as a whole. It is further understood that the above salts may form hydrates or exist in a substantially anhydrous form.

As used herein, the term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. The term “chiral center” refers to a carbon atom to which four different groups are attached. As used herein, the term “diastereomers” refers to stereoisomers which are not enantiomers. In addition, two diastereomers which have a different configuration at only one chiral center are referred to herein as “epimers”. The terms “racemate”, “racemic mixture” or “racemic modification” refer to a mixture of equal parts of enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation:

$$ⅇⅇ = \frac{{E}^{1} - {E}^{2}}{{E}^{1} + {E}^{2}} \times 100$$

wherein E¹ is the amount of the first enantiomer and E² is the amount of the second enantiomer. Thus, if the initial ratio of the two enantiomers is 50:50, such as is present in a racemic mixture, and an enantiomeric enrichment sufficient to produce a final ratio of 70:30 is achieved, the ee with respect to the first enantiomer is 40%. However, if the final ratio is 90:10, the ee with respect to the first enantiomer is 80%. An ee of greater than 90% is preferred, an ee of greater than 95% is most preferred and an ee of greater than 99% is most especially preferred. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art. In addition, the specific stereoisomers and enantiomers of compounds of formula I can be prepared by one of ordinary skill in the art utilizing well known techniques and processes, such as those disclosed by J. Jacques, et al., “Enantiomers, Racemates, and Resolutions”, John Wiley and Sons, Inc., 1981, and E. L. Eliel and S. H. Wilen, “Stereochemistry of Organic Compounds”, (Wiley-Interscience 1994), and European Patent Application No. EP-A-838448, published Apr. 29, 1998. Examples of resolutions include recrystallization techniques or chiral chromatography.

Some of the compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

The terms “R” and “S” are used herein as commonly used in organic chemistry to denote specific configuration of a chiral center. The term “R” (rectus) refers to that configuration of a chiral center with a clockwise relationship of group priorities (highest to second lowest) when viewed along the bond toward the lowest priority group. The term “S” (sinister) refers to that configuration of a chiral center with a counterclockwise relationship of group priorities (highest to second lowest) when viewed along the bond toward the lowest priority group. The priority of groups is based upon their atomic number (in order of decreasing atomic number). A partial list of priorities and a discussion of stereochemistry is contained in “Nomenclature of Organic Compounds: Principles and Practice”, (J. H. Fletcher, et al., eds., 1974) at pages 103-120.

As used herein the term “(1-6C)alkyl” refers to straight or branched, monovalent, saturated aliphatic chains of 1 to 6 carbon atoms and includes, but is not limited to, methyl, ethyl, propyl, isopropyl, butyl, isobutyl, t-butyl, pentyl, isopentyl, and hexyl. The term “(1-6C)alkyl” includes within its definition the term “(1-4C)alkyl”.

As used herein the term “(2-6C)alkenyl” refers to a straight or branched, monovalent, unsaturated aliphatic chain having from two to six carbon atoms and includes, but is not limited to, ethenyl (also known as vinyl), 1-methylethenyl, 1-methyl-1-propenyl, 1-butenyl, 1-hexenyl, 2-methyl-2-propenyl, 1-propenyl, 2-propenyl, 2-butenyl, 2-pentenyl, and the like.

As used herein the term “(1-4C)alkoxy” refers to a straight or branched alkyl chain having from one to six carbon atoms attached to an oxygen atom, and includes methoxy, ethoxy,, propoxy, isopropoxy, butoxy, t-butoxy, and the like.

The terms “halogen”, “Hal” or “halide” include fluorine, chlorine, bromine and iodine unless otherwise specified.

As used herein the term “bis(pinacolato)diboron” refers to the following structure:

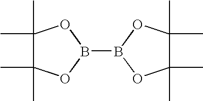

The compounds of formula I can be prepared by one of ordinary skill in the art, for example by following the various procedures set forth in the Schemes below. The reagents and starting materials are readily available to one of ordinary skill in the art, for example, see International Patent Application Publications: WO 98/33496 published Aug. 6, 1998, and WO 00/06148 and WO 00/06158, both published Feb. 10, 2000. All substituents, unless otherwise specified are as previously defined.

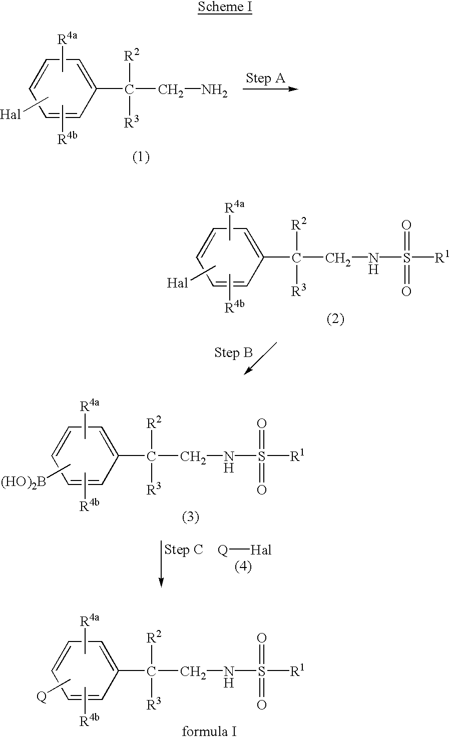

In Scheme I, step A the amine (1) is sulfonylated under conditions well known in the art to provide the sulfonamide of structure (2). For example, the amine (1) is dissolved in a suitable organic solvent. Examples of suitable organic solvents include methylene chloride, tetrahydrofuran, and the like. The solution is treated with a slight excess of a suitable base. Examples of suitable bases include triethylamine, pyridine, 1,8-diazabicyclo[5.4.0]undec-7-ene (DBU), and the like. To the stirring solution is added about 2 equivalents of a compound of formula LgSO₂R¹. The term “Lg” as used herein refers to a suitable leaving group. Examples of suitable leaving groups include, Cl, Br, and the like. Cl is the preferred leaving group. The reaction mixture is stirred for about 0.5 hours to about 16 hours. The sulfonamide (2) is then isolated and purified by techniques well known in the art, such as extraction techniques and chromatography. For example, the mixture is washed with 10% sodium bisulfate, the layers separated and the aqueous extracted with several times with a suitable organic solvent, such as methylene chloride. The organic extracts are combined, dried over anhydrous sodium sulfate, filtered, and concentrated under vacuum. The residue is then purified by chromatography on silica gel with a suitable eluent such as ethyl acetate/hexane or methanol/methylene chloride to provide the sulfonamide (2).

In Scheme I, steps B and C the sulfonamide (2) is coupled to the halide (4) under Suzuki-Type coupling reaction conditions well known to one of ordinary skill in the art. See Suziki, A., Journal of Organometallic Chemistry, 576, 147-168 (1999), and Miyaura and Suzuki, Chemical Reviews, 95, 2457-2483 (1995) for examples of such coupling reactions and conditions.

More specifically, in Scheme I, step B, the sulfonamide (2) is converted under standard conditions to the pinacol borane of structure (3). For example, sulfonamide (2) is combined with about 1.1 equivalents of bis(pinacolato)diboron, a catalytic amount of a suitable catalyst, such as [1,1′-bis(diphenylphosphino)ferrocene]dichloropalladium(II) complex with dichloromethane, and about 3 equivalents of potassium acetate in a suitable organic solvent, such as DMF. The reaction mixture is then heated at about 80° C. under an atmosphere of nitrogen for about 1 to 6 hours and then cooled to room temperature. The resulting pinacol borane (3) is then used directly in Scheme I, step C.

In Scheme I, step C, the pinacol borane (3) is treated with a halide of structure (4) under conditions well known in the art to provide the compound of formula 1. For example, about 1.5 equivalents of halide (4) is added to the above prepared solution with a catalytic amount of a suitable catalyst, such as [1,1′-bis(diphenylphosphino)ferrocene]dichloropalladium(II) complex with dichloromethane and an excess of an aqeous solution of a suitable base, such as 2M sodium carbonate. The reaction mixture is then heated at about 80° C. for about 8 to 16 hours and then cooled to room temperature. The cooled reaction mixture is diluted with a suitable organic solvent, such as diethyl ether or ethyl acetate, and washed with water. The organic layer is then dried over anhydrous magnesium sulfate, filtered and concentrated under vacuum to provide the crude compound of formula I. This crude material is then purified by techniques well known in the art, such as chromatography on silica gel with a suitable eluent, such as ethyl acetate/hexane to provide the purified compound of formula I.

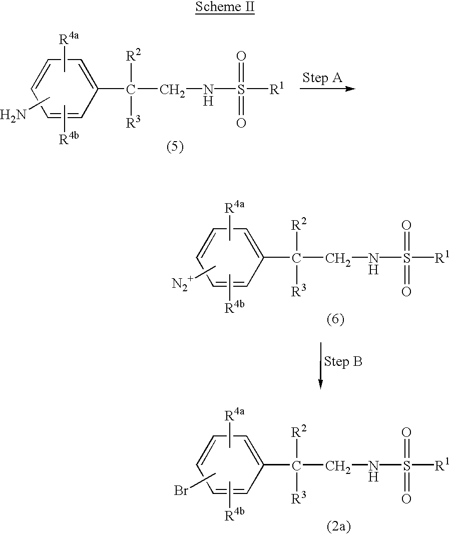

In Scheme II, the amine of structure (5) is converted under standard conditions to the bromo derivative of structure (2a). For example, see Wu and Mosher, J. Org. Chem., 51, 1904 (1986). More specifically, amine (5) is combined with an excess of 6N HCl, the solution is cooled to about 0° C. and treated with about 1.1 equivalents of an aqueous solution of sodium nitrite. The reaction mixture is allowed to stir for about 15 minutes to about one hour and urea is then added to destroy any excess sodium nitrite. The reaction mixture containing the diazonium salt (6) is then subjected to Sandmeyer Reaction conditions to provide the bromide derivative (2a). For example, see Jerry March, “Advanced Organic Chemistry: Reactions, Mechanisms, and Structure,” Fourth Edition, John Wiley & Sons, page 723, (1992) reaction number 4-25.

More specifically, the mixture containing the diazonium salt (6) is added to acetone which is at 0° C. To this solution is added a mixture of about 1.5 equivalents of CuBr and about 1.6 equivalents of LiBr. The mixture is allowed to stir for about 1 to 2 hours at 0° C.

The reaction mixture is then concentrated under vacuum, the residue dissolved in a suitable organic solvent, such as ethyl acetate, washed with water, aqueous sodium bicarbonate, brine, dried over anhydrous magnesium sulfate, fitlered, and concentrated under vacuum to provide the bromide (2a).

The following examples further illustrate the invention and represent typical syntheses of the compounds of formula I as described generally above. The reagents and starting materials are readily available to one of ordinary skill in the art. The reagents and starting materials are readily available to one of ordinary skill in the art. As used herein, the following terms have the meanings indicated: “eq” refers to equivalents; “g” refers to grams; “mg” refers to milligrams; “L” refers to liters; “mL” refers to milliliters; “μL” refers to microliters; “mol” refers to moles; “mmol” refers to millimoles; “psi” refers to pounds per square inch; “min” refers to minutes; “h” or “hr” refers to hours; “°C.” refers to degrees Celsius; “TLC” refers to thin layer chromatography; “HPLC” refers to high performance liquid chromatography; “R_{f}” refers to retention factor; “Rₜ” refers to retention time; “δ” refers to part per million down-field from tetramethylsilane; “THF” refers to tetrahydrofuran; “DMF” refers to N,N-dimethylformamide; “DMSO” refers to methyl sulfoxide; “LDA” refers to lithium diisopropylamide; “EtOAc” refers to ethyl acetate; “KOAc” refers to potassium acetate; “aq” refers to aqueous; “iPrOAc” refers to isopropyl acetate; “MTBE” refers to tert-butyl methyl ether; “methyl DAST” refers to dimethylaminosulfur trifluoride, “DAST” refers to diethylaminosulfur trifluoride, “DBU” refers to 1,8-diazabicyclo[5.4.0]undec-7-ene; as used herein “Pd(dppf)₂Cl₂ catalyst” refers to ([1,1′-bis(diphenylphosphino)ferrocene]dichloropalladium(II) complex with CH₂Cl₂; as used herein the term “NBS” refers to N-bromosuccinimde; as used herein the terms “Me”, “Et”, “Pr”, “iPr”, and “Bu” refer to methyl, ethyl, propyl, isopropyl, and butyl respectively, and “RT” refers to room temperature.

### Preparation 1

Preparation of [(2R)-2-(4-aminophenyl)propyl][(methylethyl)sulfonyl]amine.

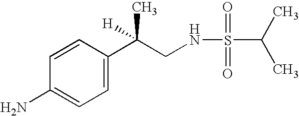

Preparation of 2-Phenyl-1-propylamine HCl.

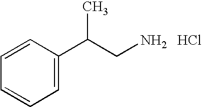

To an autoclave hydrogenation apparatus under nitrogen was charged water-wet 5% palladium on carbon (453 g), ethanol (6.36 L), 2-phenylpropionitrile (636 g, 4.85 moles) and finally concentrated (12M) hydrochloric acid (613 g, 5.6 mole). The mixture was stirred rapidly and pressurized to 75-78 psi with hydrogen. The mixture was then heated to 50-64° C. for 3 hours. ¹H NMR analysis of an aliquot showed less than 5% starting material. The reaction mixture was depressurized and filtered to afford two lots of filtrate that were concentrated under reduced pressure to ˜400 mL each. To each lot was added methyl tert-butyl ether (MTBE) (2.2 L each) and the precipitated solids were allowed to stir overnight. Each lot was filtered and the collected solids were each washed with fresh MTBE (100 mL) and dried overnight. The lots were combined to afford the intermediate title compound, 2-phenyl-1-propylamine HCl, (634.4 g, 76.2%) as a white powder.

1H NMR analysis of the free base: 1H NMR (CDCl₃, 300 MHz) δ 7.32 (m, 2H), 7.21 (m, 3H), 2.86 (m, 2H), 2.75 (m, 1H), 1.25 (d, 3H, J=6.9), 1.02 (br s, 2H).

Preparation of (2R)-2-phenylpropylamine Malate.

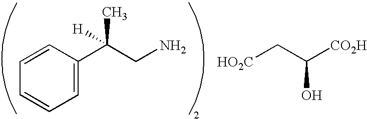

To a dry 3-Liter round bottom flask under nitrogen was charged 2-phenyl-1-propylamine HCl (317.2 g, 1.85 moles), dry ethanol (2.0 L) and NaOH beads (75.4 g, 1.89 moles) that were washed in with additional ethanol (500 mL). The mixture was stirred for 1.6 hours, and the resulting milky white NaCl salts were filtered. An aliquot of the filtrate was analyzed by gas chromatography to provide the amount of free amine, 2-phenyl-1-propylamine, (1.85 moles). A solution of L-malic acid (62.0 g, 0.462 mole, 0.25 equivalents) in ethanol (320 mL) was added dropwise to the yellow filtrate and the solution was heated to 75° C. The solution was stirred at 75° C. for 30 minutes. The heat was removed and the solution was allowed to cool slowly. The resulting thick precipitate was allowed to stir overnight. The precipitate was filtered and dried under vacuum after rinsing with ethanol (325 mL) to afford the intermediate title compound, (2R)-2-phenylpropylamine malate, (147.6 g, 39.5%) as a white crystalline solid. Chiral GC analysis of the free base, 2-phenyl-1-propylamine revealed 83.2% e.e. enriched in the R-isomer (configuration was assigned via spectrometric comparison, via chiral HPLC, with commercially available (R)-2-phenyl-1-propylamine).

¹H NMR (CDCl₃, 300 MHz) δ 7.32 (m, 2H), 7.21 (m, 3H), 2.86 (m, 2H), 2.75 (m, 1H), 1.25 (d, 3H, J=6.9), 1.02 (br s, 2H).

A slurry of (2R)-2-phenylpropylamine malate (147.1 g, 83.2% e.e.) in 1325 mL ethanol and 150 mL deionized water was heated to reflux (˜79.2° C.) until the solids went into solution. The homogeneous solution was allowed to slowly cool with stirring overnight. The precipitated white solids were cooled (0-5° C.) and filtered. The collected solids were rinsed with ethanol (150 mL) and dried at 35° C. to afford (2R)-2-phenylpropylamine malate (125.3 g, 85.2% recovery) as a white powder. Chiral GC analysis of the free base, (2R)-2-phenylpropylamine, revealed 96.7% e.e. enriched in the R-isomer.

¹H NMR (CD₃OD, 300 MHz) δ 7.32 (m, 10 H), 4.26 (dd, 1H, J=3.6, 9.9), 3.08 (m, 6H), 2.72 (dd, 1H, J=9.3, 15.3), 2.38 (dd, 1H, J=9.3, 15.6), 1.33 (d, 6H, J=6.6).

Preparation of ((2R)-2-phenylpropyl)[(methylethyl)sulfonyl]amine.

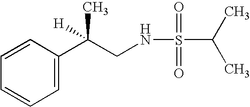

To a stirred slurry of (2R)-2-phenylpropylamine malate (200 g, 0.494 mol) in CH₂Cl₂ (1000 mL) was added 1.0 N NaOH (1050 mL, 1.05 moles). The mixture was stirred at room temperature for 1 hour and the organic phase was separated and gravity filtered into a 3.0 L round-bottom flask with a CH₂Cl₂ rinse (200 mL). The resulting free base, (2R)-2-phenylpropylamine, was dried via azeotropic distillation. Accordingly, the clear filtrate was concentrated to 600 mL at atmospheric pressure via distillation through a simple distillation head. Heptane (1000 mL) was added and the solution was concentrated again at atmospheric pressure to 600 mL using a nitrogen purge to increase the rate of distillation. The final pot temperature was 109° C.

The solution was cooled to room temperature under nitrogen with stirring to give a clear, colorless heptane solution (600 mL) of (2R)-2-phenylpropylamine. To this solution was added 4-dimethylaminopyridine (6.04 g, 0.0494 mol), triethylamine (200 g, 1.98 moles), and CH₂Cl₂ (500 mL). The mixture was stirred at room temperature until a clear solution was obtained. This solution was cooled to 5° C. and a solution of isopropylsulfonyl chloride (148 g, 1.04 moles) in CH₂Cl₂ (250 mL) was added dropwise with stirring over 2 hrs. The mixture was allowed to warm gradually to room temperature over 16 h. GC analysis indicated complete consumption of the (2R)-2-phenylpropylamine starting material.

The stirred mixture was cooled to 8° C. and 2 N HCl (500 mL) was added dropwise. The organic phase was separated and extracted with water (1×500 mL) and saturated NaHCO₃ (1×500 mL). The organic phase was isolated, dried (Na₂SO₄), and gravity filtered. The filtrate was concentrated under reduced pressure to provide the intermediate title compound, ((2R)-2-phenylpropyl)[(methylethyl)sulfonyl]amine, (230 g, 96%) as a pale yellow oil. ¹H NMR (CDCl₃, 300 MHz) δ 7.34 (m, 2H), 7.23 (m, 3H), 3.89 (br t, 1H, J=5.4), 3.36 (m, 1H), 3.22 (m, 1H), 3.05 (m, 1H), 2.98 (m, 1H), 1.30 (d, 3H, J=7.2), 1.29 (d, 3H, J=6.9), 1.25 (d, 3H, J=6.9).

Preparation of [(2R)-2-(4-aminophenyl)propyl][(methylethyl)sulfonyl]amine p-toluenesulfonate.

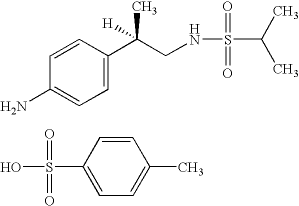

To a round-bottom flask equipped with stir rod, thermocouple and nitrogen purge at 25° C., was charged ((2R)-2-phenylpropyl)[(methylethyl)sulfonyl]amine (5.00 g, 0.0207 mol), trifluoroacetic acid (15 mL), dichloromethane (1.2 mL) and heptane (8 mL). The mixture was cooled to −5° C. and 98% fuming nitric acid (1.60 g, 0.0249 mol) was added dropwise. The reaction mixture was stirred at −5 to +5° C. for 3-5 hours and then warmed to 20-25° C. The reaction was allowed to stir until GC analysis revealed that ((2R)-2-phenylpropyl)[(methylethyl)sulfonyl]amine is less then 1% (area %).

The reaction mixture was then diluted with dichloromethane (20 mL) and diionized water (20 mL), and the mixture was transferred to a suitably sized 3-neck bottom outlet round-bottom flask. The mixture was stirred for 10-15 minutes. The aqueous phase was separated, extracted with dichloromethane (1×20 mL), and the organic phases were combined. To the organic phase was added water (15 mL), 10% NaOH (10 mL), and the pH was adjusted to 6.5-7.5 with saturated sodium carbonate. After 10-15 minintes of stirring, the organic layer was separated and concentrated to an oil under reduced pressure (25-35° C.). The oil containing the mixture of [(2R)-2-(4-nitrophenyl)propyl][(methylethyl)sulfonyl]amine, [(2R)-2-(3-nitrophenyl)propyl][(methylethyl)sulfonyl]amine, and [(2R)-2-(2-nitrophenyl)propyl][(methylethyl)sulfonyl]amine, was diluted with ethanol and was transferred to a Parr bottle containing 1.25 g of 5% Pd on C (rinsed in with 5 mL of THF) under nitrogen (total ethanol=45 mL). The reaction mixture was hydrogenated for 16-20 hours at 20-25° C. until the GC area % of [(2R)-2-(4-aminophenyl)propyl][(methylethyl)sulfonyl]amine was greater than 70%. The reaction mixture was filtered through Hyflo followed by an ethanol rinse (25 mL).

The oil was diluted with THF (35 mL) and p-toluenesulfonic acid monohydrate (3.94 g, 0.0207 mol) was added with stirring at 20-25° C. When the solids completely dissolved, MTBE (22 mL) was added and the slurry was stirred for 1-2 hours. The slurry was filtered and the cake was rinsed three times with a 3:7 (v/v) solution of MBTE and THF. This process afforded the intermediate title compound, [(2R)-2-(4-aminophenyl)propyl][(methylethyl)sulfonyl]amine p-toluenesulfonate, in 53.5% yields as an off white powder. Chiral analysis of the freebase, [(2R)-2-(4-aminophenyl)propyl][(methylethyl)sulfonyl]amine, obtained extractively from [(2R)-2-(4-aminophenyl)propyl][(methylethyl)sulfonyl]amine p-toluenesulfonate, showed % e.e. of 99.5%.

¹H NMR (CD₃OD, 300 MHz) δ 7.70 (d, 2H, J=8.4), 7.43 (d, 2H, J=8.4), 7.33 (d, 2H, J=8.4), 7.23 (d, 2H, J=7.8), 3.22 (m, 2H), 3.08 (quint, 1H, J=6.9), 2.99 (q, 1H, J=6.9), 1.29 (d, 3H, J=6.6), 1.23 (d, 3H, J=6.6).

Preparation of Final Title Compound.

To a suspension of [(2R)-2-(4-aminophenyl)propyl][(methylethyl)sulfonyl]amine p-toluenesulfonate (41.2 g, 0.0961 mol) in CH₂Cl₂ (300 mL) was added saturated aqueous NaHCO₃ until the pH of the aqueous phase was 6.5. The phases were separated and the organic phase was washed with 5% NaHCO₃ (2×100 mL), H₂O (100 mL), and concentrated to provide [(2R)-2-(4-aminophenyl)propyl][(methylethyl)sulfonyl]amine as an oil. After diluting the oil with diethyl ether (50 mL), crystallization began after 10 min. Caution: Heat of crystallization caused ether to boil. After the exotherm subsided (45 minutes), the suspension was filtered, and the filter cake was washed with diethyl ether (2×20 mL), and dried under reduced pressure to afford the final title compound, [(2R)-2-(4-aminophenyl)propyl][(methylethyl)sulfonyl]amine, (21.7 g, 88.1%). ¹H NMR (CDCl₃, 300 MHz) δ 7.00 (d, 2H, J=8.1); 6.66 (d, 2H, J=8.4), 3.83 (m, 1H), 3.65 (br s, 2H), 3.31 (m, 1H), 3.09 (m, 2H), 2.85 (m, 1H), 1.30 (d, 3H, J=7.2), 1.26 (d, 3H, J=6.9), 1.24 (d, 3H, J=6.9).

### Preparation 2

Preparation of [(2S)-2-(4-aminophenyl)propyl][(methylethyl)sulfonyl]amine.

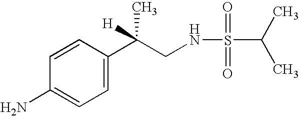

The title compound is prepared from (2S)-2-phenylpropylamine in a manner analogous to the procedure described in preparation 1.

### Preparation 3

Preparation of [(2R)-2-(4-iodophenyl)propyl][(methylethyl)sulfonyl]amine.

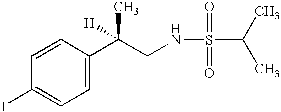

Preparation of 2-Phenyl-1-propylamine HCl.

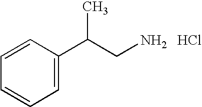

To an autoclave hydrogenation apparatus under nitrogen was charged water-wet 5% palladium on carbon (453 g), ethanol (6.36 L), 2-phenylpropionitrile (636 g, 4.85 moles) and finally concentrated (12M) hydrochloric acid (613 g, 5.6 mole). The mixture was stirred rapidly and pressurized to 75-78 psi with hydrogen. The mixture was then heated to 50-64° C. for 3 hours. ¹H NMR analysis of an aliquot showed less than 5% starting material. The reaction mixture was depressurized and filtered to afford two lots of filtrate that were concentrated under reduced pressure to ˜400 mL each. To each lot was added methyl tert-butyl ether (MTBE) (2.2 L each) and the precipitate solids were allowed to stir overnight. Each lot was filtered and the collected solids were each washed with fresh MTBE (100 mL) and dried overnight. The lots were combined to afford 2-phenyl-1-propylamine HCl (634.4 g, 76.2%) as a white powder. ¹H NMR analysis of the free base: ¹H NMR (CDCl₃, 300 MHz) δ 7.32 (m, 2H), 7.21 (m, 3H), 2.86 (m, 2H), 2.75 (m, 1H), 1.25 (d, 3H, J=6.9), 1.02 (br s, 2H).

Preparation of (2R)-2-phenylpropylamine Malate.

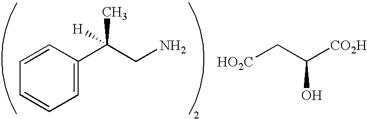

To a dry 3-Liter round bottom flask under nitrogen was charged 2-phenyl-1-propylamine HCl (317.2 g, 1.85 moles), dry ethanol (2.0 L) and NaOH beads (75.4 g, 1.89 moles) that were washed in with additional ethanol (500 mL). The mixture was stirred for 1.6 hours, and the resulting milky white NaCl salts were filtered. An aliquot of the filtrate was analyzed by gas chromatography to provide the amount of free amine, 2-phenyl-1-propylamine, (1.85 moles). A solution of L-malic acid (62.0 g, 0.462 mole, 0.25 equivalents) in ethanol (320 mL) was added dropwise to the yellow filtrate and the solution was heated to 75° C.. The solution was stirred at 75° C. for 30 minutes. The heat was removed and the solution was allowed to cool slowly. The resulting thick precipitate was allowed to stir overnight. The precipitate was filtered and dried under vacuum after rinsing with ethanol (325 mL) to afford (2R)-2-phenylpropylamine malate (147.6 g, 39.5%) as a white crystalline solid. Chiral GC analysis of the free base, 2-phenyl-1-propylamine revealed 83.2% e.e. enriched in the R-isomer (configuration was assigned via spectrometric comparison with commercial 2-phenyl-1-propylamine) ¹H NMR (CDCl₃, 300 MHz) δ 7.32 (m, 2H), 7.21 (m, 3H), 2.86 (m, 2H), 2.75 (m, 1H), 1.25 (d, 3H, J=6.9), 1.02 (br s, 2H).

A slurry of (2R)-2-phenylpropylamine malate (147.1 g, 83.2% e.e.) in 1325 mL ethanol and 150 mL deionized water was heated to reflux (˜79.2° C.) until the solids went into solution. The homogeneous solution was allowed to slowly cool with stirring overnight. The precipitated white solids were cooled (0-5° C.) and filtered. The collected solids were rinsed with ethanol (150 mL) and dried at 35° C. to afford (2R)-2-phenylpropylamine malate (125.3 g, 85.2% recovery) as a white powder. Chiral GC analysis of the free base, (2R)-2-phenylpropylamine, revealed 96.7% e.e. enriched in the R-isomer.

¹H NMR (CD₃OD, 300 MHz) δ 7.32 (m, 10 H), 4.26 (dd, 1H, J=3.6, 9.9), 3.08 (m, 6H), 2.72 (dd, 1H, J=9.3, 15.3), 2.38 (dd, 1H, J=9.3, 15.6), 1.33 (d, 6H, J=6.6).

Preparation of ((2R)-2-phenylpropyl)[(methylethyl)sulfonyl]amine.

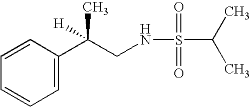

To a stirred slurry of (2R)-2-phenylpropylamine malate (200 g, 0.494 mol) in CH₂Cl₂ (1000 mL) was added 1.0 N NaOH (1050 mL, 1.05 moles). The mixture was stirred at room temperature for 1 hour and the organic phase was separated and gravity filtered into a 3.0 L round-bottom flask with a CH₂Cl₂ rinse (200 mL). The resulting free base, (2R)-2-phenylpropylamine, was dried via azeotropic distillation. Accordingly, the clear filtrate was concentrated to 600 mL at atmospheric pressure via distillation through a simple distillation head. Heptane (1000 mL) was added and the solution was concentrated again at atmospheric pressure to 600 mL using a nitrogen purge to increase the rate of distillation. The final pot temperature was 109° C.

The solution was cooled to room temperature under nitrogen with stirring to give a clear, colorless heptane solution (600 mL) of (2R)-2-phenylpropylamine. To this solution was added 4-dimethylaminopyridine (6.04 g, 0.0494 mol), triethylamine (200 g, 1.98 moles), and CH₂Cl₂ (500 mL). The mixture was stirred at room temperature until a clear solution was obtained. This solution was cooled to 5° C. and a solution of isopropylsulfonyl chloride (148 g, 1.04 moles) in CH₂Cl₂ (250 mL) was added dropwise with stirring over 2 hrs. The mixture was allowed to warm gradually to room temperature over 16 h. GC analysis indicated complete consumption of the (2R)-2-phenylpropylamine starting material.

The stirred mixture was cooled to 8° C. and 2 N HCl (500 mL) was added dropwise. The organic phase was separated and extracted with water (1×500 mL) and saturated NaHCO₃ (1×500 mL). The organic phase was isolated, dried (Na₂SO₄), and gravity filtered. The filtrate was concentrated under reduced pressure to provide ((2R)-2-phenylpropyl)[(methylethyl)sulfonyl]amine (230 g, 96%) as a pale yellow oil. ¹H NMR (CDCl₃, 300 MHz) δ 7.34 (m, 2H), 7.23 (m, 3H), 3.89 (br t, 1H, J=5.4), 3.36 (m, 1H), 3.22 (m, 1H), 3.05 (m, 1H), 2.98 (m, 1H), 1.30 (d, 3H, J=7.2), 1.29 (d, 3H, J=6.9), 1.25 (d, 3H, J=6.9).

Preparation of Final Title Compound.

A stirred room temperature solution of ((2R)-2-phenylpropyl)[(methylethyl)sulfonyl]amine (37.1 g, 0.154 mol) in glacial acetic acid (185 mL) was treated with concentrated H₂SO₄ (16.0 g, 0.163 mol), added dropwise in a slow stream, followed by a H₂O rinse (37 mL). To this solution (˜30° C.) was added H₅IO₆ (8.29 g, 0.0369 mol), followed by iodine (17.9 g, 0.0707 mol). The resulting reaction mixture was heated and allowed to stir for 3 h at 60° C. After HPLC analysis verified the consumption of starting material, the reaction mixture was cooled to 30° C and a 10% aqueous solution of NaHSO₃ (220 mL) was added dropwise while maintaining the temperature between 25° C. and 30 0 C. The mixture crystallized to a solid mass upon cooling to 0-5° C.

The solids were suction filtered and rinsed with H₂O to afford 61.7 g of crude solids that were redissolved into warm MTBE (500 mL). This solution was extracted with H₂O (2×200 mL) and saturated NaHCO₃ (1×200 mL) and the organic phase was dried (MgSO₄), filtered, and concentrated under reduced pressure to ˜200 mL. Heptane (100 mL) was added dropwise to the product solution with slow stirring until crystallization commenced. An additional 100 mL of heptane was added and the resulting suspension was allowed to stir slowly overnight at room temperature. The mixture was then cooled (0° C.), filtered, and the collected solids were rinsed with heptane. The solids were then air-dried to afford the final title compound, [(2R)-2-(4-iodophenyl)propyl][(methylethyl)sulfonyl]amine (33.7 g, 59.8%) as a white powder. Chiral Chromatography of this lot indicated 100% e.e.

¹H NMR (CDCl₃, 300 MHz) δ 7.66 (d, 2H, J=8.1), 6.98 (d, 2H, J=8.4), 3.86 (br t, 1H, J=5.1), 3.33 (m, 1H), 3.18 (m, 1H), 3.06 (m, 1H), 2.92 (m, 1H), 1.30 (d, 3H, J=6.6), 1.27 (d, 6H, J=6.6).

### Preparation 4

Preparation of [(2S)-2-(4-iodophenyl)propyl][(methylethyl)sulfonyl]amine.

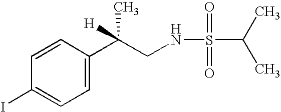

To a stirred solution of ((2S)-2-phenylpropyl)[methylethyl)sulfonyl]amine (5.00 g, 20.7 mmol) in glacial HOAc (25.0 mL) was added H₂SO₄ (1.25 mL, 18.0 mmol) dropwise followed by a H₂O (10 mL) rinse. To the resulting solution was added H₅IO₆ (1.14 mL, 5.00 mmol) followed by I₂ (2.42 g, 9.5 mmol). The reaction mixture was stirred for 3 h at 60° C. After cooling to ambient temperature, an aqueous solution of NaHSO₃ (30 mL) was added dropwise. The resulting mixture was poured into Et₂O (100 mL), extracted twice with H₂O and dried (Na₂SO₄). Filtration and concentration of the filtrate gave the the title compond as a yellow oil which formed white crystals 3.70 g.

### Preparation 5

Preparation of 6-Bromo-3,4-dihydro-1H-quinolin-2-one.

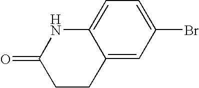

To a stirred solution of 3,4-dihydro-1H-quinolin-2-one (0.735 g, 5.0 mmol) in dry DMF (20.0 mL) under N₂ at ambient temperature was added N-bromosuccimide (0.93 g, 5.2 mmol) portionwise. The solution was stirred under N₂ overnight then the orange mixture was poured into H₂O (200 mL) and the precipitated solid was extracted into ether(100 mL). The ether was separated extracted with of H₂O, (4×150 mL) washed with brine and dried(MgSO₄). Filtration and evaporation in vacuo gave the title compound (0.70 g, 62%) as a light tan powder.

¹H NMR(CDCl₃) δ 2.60 (2H, m), 2.90 (2H, m), 6.65 (1H, d), 7.2-7.3 (2H, m), 8.9(1H, br s).

### Preparation 6

Preparation of 6-(4,4,5,5-Tetramethyl-[1,3,2]dioxaborolan-2-yl)3,4-dihydro-1H-quinolin-2-one.

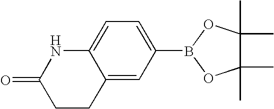

6-Bromo-3,4-dihydro-1H-quinolin-2-one (0.35 g, 1.35 mmol) was combined with pinacol borane (0.30 g, 2.33 mmol), [1,1′-bis(diphenylphosphino)ferrocene]dichloropalladium(II) complex with dichloromethane (0.04 g, 0.05 mmol) and triethylamine (0.65 mL, 4.65 mmol) in dry acetonitrile (7 mL) and heated at reflux under N₂ for 4 hours then cooled to ambient temperature. The reaction mixture was dumped into diethyl ether and washed with water and saturated NaCl, dried (MgSO₄), filtered, and concentrated to give the title compound (0.44 g) as a light yellow oil which was used without purification.

### Example 1

Preparation of 6-[4-(1-methyl-2-{[(methylethyl)sulfonyl]amino}ethyl)phenyl]-3-hydrobenzothiazol-2-one.

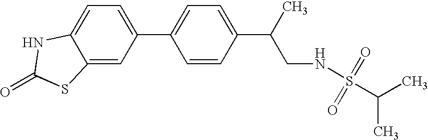

Preparation of [2-(4-bromophenyl)propyl][(methylethyl)sulfonyl]amine.

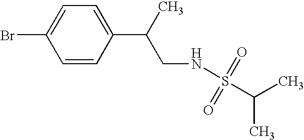

The title compound can be prepared in a manner analogous to the procedure set forth in Example 39 of WO 98/33496 published Aug. 6, 1998. For example, to a −15° C. solution of 4-bromo-acetophenone (50.0 g, 251.2 mmol) and tosylmethyl isocyanide (49.0 g, 251.2 mmol) in 800 mL of dry dimethoxyethane was added a hot solution of potassium tert-butoxide (50.7 g, 452.2 mmol) in 230 mL of tert-butyl alcohol dropwise at a rate to maintain the temperature below 0° C. The reaction was stirred at −5° C. for 45 min after addition was complete. The cooling bath was removed and the reaction stirred for 2.5 h more. The mixture was concentrated in vacuo to a volume of 200 mL and diluted with 500 mL of water. The aqueous mixture was extracted four times with diethyl ether, and the combined organic portions were dried (MgSO₄), filtered, and concentrated in vacuo. The residue was dissolved in 55 mL of tetrahydrofuran and heated to reflux. To the refluxing solution was added slowly dropwise 27.6 mL (276.3 mmol) of 10.0 M borane-dimethylsulfide complex. Refluxing was continued for 20 min after addition was complete. The mixture was cooled to ambient temperature and methanol saturated with hydrogen chloride was added very slowly until pH 2 was achieved. The mixture was concentrated in vacuo and the residue was dissolved in methanol and concentrated in vacuo again. The solid residue was suspended in 125 mL of ethanol, filtered, rinsed with ethanol then diethyl ether. The white solid was dried in vacuo to afford 2-(4-bromophenyl)propylamine hydrochloride (25.4 g, 40%). The filtrate was concentrated in vacuo and suspended in diethyl ether. The solid was filtered, rinsed with diethyl ether and dried in vacuo to afford another portion of 2-(4-bromophenyl)propylamine hydrochloride (15.6 g, 25%).

Then, a solution of 2-(4-bromophenyl)propylamine hydrochloride (15.0 g, 59.9 mmol) and triethylamine (18.4 mL, 131.8 mmol) in 150 mL of dichloromethane was stirred 20 min at room temperature, then cooled to 0° C. and treated dropwise over 5 min with 2-propylsulfonyl chloride (8.1 mL, 71.9 mmol) in 10 mL of dichloromethane. After stirring overnight at room temperature, the reaction was washed once with 200 mL of 10% aqueous sodium bisulfate, the layers separated and the aqueous layer extracted twice with 100 mL each of dichloromethane. The combined organic extracts were dried dried (MgSO₄), filtered, and concentrated in vacuo. Chromatography (500 g of silica gel, 30% ethyl acetate/hexane) of the residue afforded 11.0 g (57%) of the intermediate title compound, [2-(4-bromophenyl)propyl][(methylethyl)sulfonyl]amine.

Preparation of Final Title Compound.

Scheme I, step C: [2-(4-Bromophenyl)propyl][(methylethyl)sulfonyl]amine (0.93 g, 2.90 mmol) was combined with bis(pinacolato)diboron (0.81 g, 3.19 mmol), [1,1′-bis(diphenylphosphino)ferrocene]dichloropalladium(II) complex with dichloromethane (0.07 g, 0.09 mmol) and potassium acetate (0.85 g, 8.70 mmol) in dry dimethylformamide (20 mL) and heated at 80° C. under N₂ for 3 hours then cooled to ambient temperature. 6-Bromo-2-benzothiazolinone (1.00 g, 4.35 mmol), 2 M Na₂CO₃ solution (7.3 mL, 14.50 mmol) and more [1,1′-bis(diphenylphosphino)ferrocene]dichloropalladium(II) complex with dichloromethane (0.07 g, 0.09 mmol) were added and the reaction was heated at 80° C. overnight. The reaction mixture was cooled to ambient temperature and diluted with diethyl ether and extracted with water. The organic layer was separated and dried (MgSO₄) and concentrated to give a tan solid which was purified by silica gel chromatography eluting with 30% ethyl acetate/hexanes to give the final title compound, 6-[4-(1-methyl-2-{[(methylethyl)sulfonyl]amino}ethyl)phenyl]-3-hydrobenzothiazol-2-one, (0.47 g, 42%) as an off-white solid. The title compound was recrystallized from diethyl ether. ¹HNMR (CDCl₃): δ 1.15 (9 H, m), 2.92 (1H, m), 3.09 (3H, m), 7.01 (2H, d), 7.21 (1H, d), 7.40 (2H, m), 7.57 (1H, d), 7.81 (1H, d), 12.01 (1H, br. s).

### Example 2

Preparation of 6-[4-(1-methyl-2-{[(methylethyl)sulfonyl]amino}ethyl)phenyl]-3-hydrobenzoxazol-2-one.

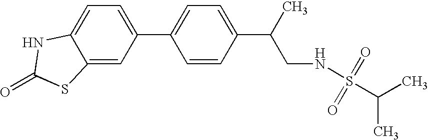

Preparation of 6-Bromo-3H-benzoxazol-2-one.

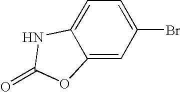

To a stirred suspension of 3H-benzoxazol-2-one (1.35 g, 10 mmol) in acetonitrile (23 mL) at −15° C. was added portionwise NBS (2.00 g, 11.0 mmol). Following complete addition of NBS the mixture was stirred at −15 to 0° C. for 3 h then allowed to warm to ambient temperature and stirred overnight. The solvent was evaporated in vacuo and the residue was partitioned between CH₂Cl₂/H₂O precipitating the intermediate title compound, 6-bromo-3H-benzoxazol-2-one, (0.67 g, 31%) as a brown solid. ¹HNMR(CDCl₃) δ 7.0 (1H, d), 7.15 (1H.,d), 7.3 (1H, s), 11.9 (1H, s).

Preparation of Final Title Compound.

Scheme I, step C: The final title compound was prepared from [2-(4-bromophenyl)propyl][(methylethyl)sulfonyl]amine (0.50 g, 1.56 mmol, prepared in example 1) and 6-bromo-3H-benzooxazol-2-one (0.50 g, 2.34 mmol) in a manner analogous to the procedure set forth in Example 1. Purification by silica gel chromatography eluting with 1:1 ethyl acetate/hexanes provided the final title compound, 6-[4-(1-methyl-2-{[(methylethyl)sulfonyl]amino}ethyl)phenyl]-3-hydrobenzoxazol-2-one, (0.112 g, 19%) as an off-white powder. ¹HNMR (CDCl₃): δ 1.29 (9 H, m), 2.87-3.38 (4 H, m), 6.86 (1H, m), 6.94 (1H, m), 7.06 (1H, m), 7.20 (1H, m), 7.28 (1H, m), 7.35 (1H, M), 7.46 (1H, m).

### Example 3

Preparation of 5-[4-(1-methyl-2-{[(methylethyl)sulfonyl]amino}ethyl)phenyl]indolin-2-one.

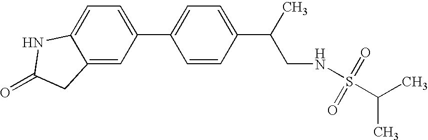

Preparation of 5-Bromo-1,3-dihydro-indol-2-one.

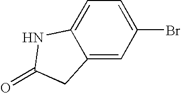

5-Bromo-1,3-dihydro-indol-2-one can be prepared following the procedure described by Sun et al., J. Med. Chem., 41, 2588-2603 (1998). For example, to a stirred suspension of oxindole(1.30 g, 10 mmol) in dry acetonitrile(22.0 mL) at −10° C. was added portionwise recrystallized NBS (2.00 g, 11.0 mmol) and the resulting suspension was stirred at −10 to 0° C. for 3 hours. The suspension was allowed to warm to ambient temperature and the mixture was filtered to give a white solid which was recrystallized (EtOH) to provide the intermediate title compound (1.47, 43%) as a slightly pink solid; mp 212-214° C.

Preparation of Final Title Compound.

Scheme I, step C: [2-(4-Bromophenyl)propyl][(methylethyl)sulfonyl]amine (1.00 g, 3.15 mmol, prepared in example 1), bis(pinacolato)diboron (0.881 g, 3.47 mmol), PdCl₂(dppf).CH₂Cl₂ (0.073 g, 0.09 mmol) and potassium acetate (0.927 g, 9.45 mmol) were heated and stirred in dry DMF (25 mL) for 3 hours under N₂. The reaction mixture was allowed to cool to ambient temperature and 5-bromo-1,3-dihydro-indol-2-one (1.00 g, 4.72 mmol), PdCl₂(dppf).CH₂Cl₂ (0.073 g, 0.09 mmol), and 2M Na₂CO₃ (7.9 mL, 15.8 mmol) were added respectively. The resulting mixture was stirred and heated at 80° C. for 6 hours. The reaction mixture was allowed to cool to ambient temperature and poured into Et₂O (100 mL) and extracted with H₂O. The aqueous layer was separated and extracted with EtOAc. The organic layers were combined, washed with brine and dried (MgSO₄). Filtration and concentration in vacuo gave a brown oil which when chromatographed on the Chromatron (Harrison Research, Palo Alto, Calif. 94306) using a 4 mm plate and eluting with a gradient EtOAc/hexane 1:1 to 4:1 system provided the final title compound, 5-[4-(1-methyl-2-{[(methylethyl)sulfonyl]amino}ethyl)phenyl]indolin-2-one, (0.530 g, 45%) as a white crystalline solid, mp 170-171° C.

Anal. Calcd: C, 64.69; H, 6.49; N, 7.52

Found:C, 64.34; H, 6.52; N, 7.40

### Example 4

Preparation of 5-[4-((1R)-1-methyl-2-{[(methylethyl)sulfonyl]amino}ethyl)phenyl]indolin-2-one.

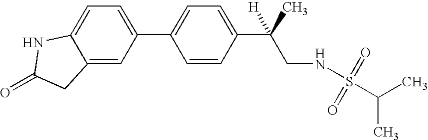

Preparation of [(2R)-2-(4-bromophenyl)propyl][(methylethyl)sulfonyl]amine.

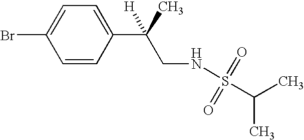

The title compound is prepared from [(2R)-2-(4-aminophenyl)propyl][(methylethyl)sulfonyl]amine in a manner analogous to the procedure set forth in Example 5.

Preparation of Final Title Compound.

Scheme I, step C: The final title compound, 5-[4-((1 R)-1-methyl-2-{[(methylethyl)sulfonyl]amino}ethyl)phenyl]indolin-2-one, (0.200 g, 17%, crystalline solid, mp 184-185° C., MS 372.9; 371.9) was prepared in a manner analogous to the procedure set forth in example 3 from [(2R)-2-(4-bromophenyl)propyl][(methylethyl)sulfonyl]amine (1.0 g, 3.15 mmol).

### Example 5

Preparation of 5-[4-((1S)-1-methyl-2-{[(methylethyl)sulfonyl]amino}ethyl)phenyl]indolin-2-one.

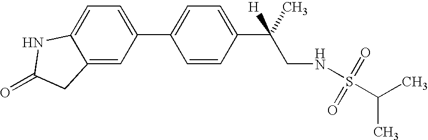

Preparation of [(2S)-2-(4-bromophenyl)propyl][(methylethyl)sulfonyl]amine.

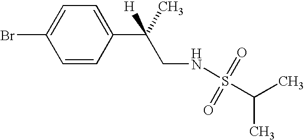

Scheme II, step A: Within a 50 mL, 3-neck flask was placed [(2S)-2-(4-aminophenyl)propyl][(methylethyl)sulfonyl]amine (317 mg, 1.24 mmoles) followed by 6N HCl (4.9 mL, 29.3 mmoles). The resulting clear, yellow solution was cooled to 0° C. and was stirred for 14 min. a solution comprised of sodium nitrite (102 mg, 1.48 mmoles) in water (2 mL) was added dropwise to the reaction mixture and the solution became a more pale yellow. After 17 minutes, urea (97 mg, 1.6 mmoles) was added to destroy excess nitrite and the solution was stirred an additional 24 minutes.

Scheme II, step B: The mixture was transferred by pasteur pipet to a 0° C. acetone solution (20 mL) within another 50 mL 3-neck round-bottom flask. To this solution was added a mixture of CuBr (265 mg, 1.85 mmoles) and LiBr (172 mg, 1.98 moles) in two portions. A mild gas evolution was seen. The mixture was allowed to stir for an hour at 0° C. TLC analysis (100% EtOAc) showed complete conversion to the aryl bromide.

The reaction mixture was concentrated by rotovap, re-diluted with EtOAc and washed with water (2×50 mL), aqueous sodium bicarbonate (1×50 mL) and brine (1×50 mL). The organic phase was separated, dried over anhydrous magnesium sulfate, fitlered, and concentrated to a tan oil which spontaneously crystallized to provide the intermediate title compound, [(2R)-2-(4-bromophenyl)propyl][(methylethyl)sulfonyl]amine, (367 mg, 92%) as a tan powder.

Preparation of Final Title Compound.

Scheme I, step C: The final title compound, 5-[4-((1S)-1-methyl-2-{[(methylethyl)sulfonyl]amino}ethyl)phenyl]indolin-2-one, (mp 170-171° C.) was prepared in a manner analogous to the procedure set forth in example 3 from [(2R)-2-(4-bromophenyl)propyl][(methylethyl)sulfonyl]amine (1.0 g, 3.15 mmol).

### Example 6

Preparation of 6-[4-(1-methyl-2-{[(methylethyl)sulfonyl]amino}ethyl)phenyl]indolin-2-one.

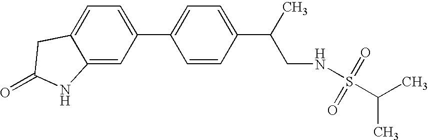

Preparation of {4-{1-Methyl-2-(propane-2-sulfonylamino)ethyl]-3-nitro-biphenyl-4-yl}-acetic Acid Ethyl Ester.

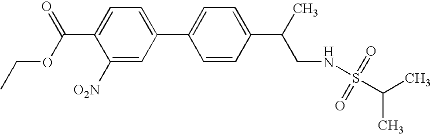

4-Bromo-2-nitrophenyl acetic acid ethyl ester (0.172 g, 0.60 mmol) was added to a stirred mixture of 2-[4-(4,4,5,5-tetramethyl-[1,3,2] dioxaborolan-2yl)-phenyl]propyl-2-propanesulfonamide (0.200 g, 0.544 mmol), PdCl₂(dppf).CH₂Cl₂ (0.042 g, 0.051 mmol) and Cs₂CO₃ (0.400 g, 1.2 mmol) which was stirred and heated at 96° C. under nitrogen for 6 h. The reaction mixture was then stirred at ambient temperature for 72 h. It was diluted with EtOAc (100 mL) and filtered through Celite®. The filtrate was washed with brine,dried (MgSO₄), and filtered through Celite®. Evaporation of the filtrate and chromatography on the Chromatron using a 4 mm plate and eluting with EtOAc/hexanes (4:6) provided the intermediate title compound, {4-{1-methyl-2-(propane-2-sulfonylamino)ethyl]-3-nitro-biphenyl-4-yl}-acetic acid ethyl ester, (0.163 g, 67%) as a viscous yellow oil. MS:447.2(M−1).

Preparation of {4-{1-Methyl-2-(propane-2-sulfonylamino)ethyl]-3-nitro-biphenyl-4-yl}-acetic Acid.

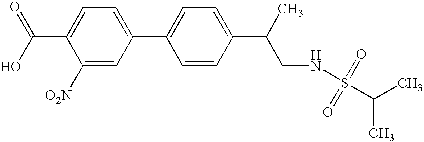

To a stirred solution of {4-{1-methyl-2-(propane-2-sulfonylamino)ethyl]-3-nitro-biphenyl-4-yl}-acetic acid ethyl ester (0.624 g, 2.17 mmol) in EtOH (5.0 mL) under nitrogen was added a solution of NaOH (1.00 g) in H₂O (15.0 mL) and the resulting dark brown mixture was stirred at ambient temperature for 72 h. The resulting orange solution was diluted with H₂O (100 mL), acidified with aqueous HCl, and extracted with EtOAc. The EtOAc layer was separated, washed with brine, dried (MgSO₄,) and filtered. Evaporation of the solvent in vacuo provided the intermediate title compound, {4-{1-methyl-2-(propane-2-sulfonylamino)ethyl]-3-nitro-biphenyl-4-yl}-acetic acid, (0.296 g, 99%) as a yellow oil which slowly crystallized; mp 92-93° C.

Preparation of Final Title Compound.

To a stirred solution of {4-{1-methyl-2-(propane-2-sulfonylamino)ethyl]-3-nitro-biphenyl-4-yl}-acetic acid (0.300 g, 0.71 mmol) dissolved in 50% H₂SO₄ (3 mL)/EtOH (4.0 mL) at 90° C., was added Zn (0.086 g, 2.84 mmol) over 30 min. Heating was continued for 2 h, then the mixture was allowed to cool to ambient temperature, and extracted with EtOAc. The organic layer was separated and extracted with 5% NaHCO₃, washed with brine, dried (MgSO₄), filtered, and concentrated in vacuo to a brown oil. Chromatography on the Chromatron gave the final title compound, 6-[4-(1-methyl-2-{[(methylethyl)sulfonyl]amino}ethyl)phenyl]indolin-2-one, as a as a light tan solid; mp 182-184° C.

### Example 7

Preparation of 6-[4-((1R)-1-methyl-2-{[(methylethyl)sulfonyl]amino}ethyl)phenyl]indolin-2-one.

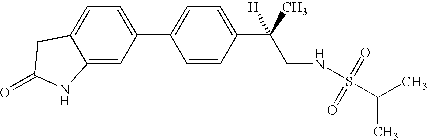

Preparation of 6-Bromo-1,3-dihydro-indol-2-one.

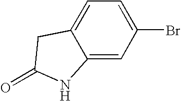

To a stirred solution of 4-bromo-2-nitrophenylacetic acid (1.00 g, 3.85 mmol) dissolved in 50% H₂SO₄ (8.2 mL)/EtOH (10.9 mL) was added Zn dust (1.01 g, 15.38 mmol) at 90° C. under nitrogen. The reaction was then treated in a manner analogous to the cyclization described above in example 6 to provide the intermediate title compound, 6-bromo-1,3-dihydro-indol-2-one, (0.512 g, 63%); mp 167-169° C.

Preparation of Final Title Compound.

Scheme I, step C: [(2R)-2-(4-Iodophenyl)propyl][(methylethyl)sulfonyl]amine (0.787 g, 2.14 mmol) bis(pinacolate) diboron (0.699 g,2.36 mmol) PdCl₂(dppf).CH₂Cl₂ (0.052 g, 0.064 mmol) and KOAc (0.630 g, 6.42 mmol) were combined and heated at 80° C. under nitrogen for 3 h. The mixture was allowed to cool to ambient temperature and 6-bromo-1,3-dihydro-1-indol-2-one (0.500 g,2.36 mmol), PdCl₂(dppf).CH₂Cl₂ (0.052 g, 0.064 mmol), 2M Na₂CO₃ (5.3 mL, 10.7 mmol) respectively were added. The resulting mixture was heated and stirred at 80° C. overnight and worked up in a manner analogous to the procedure described in example 3 to provide the final title compound, 6-[4-((1R)-1-methyl-2-{[(methylethyl)sulfonyl]amino}ethyl)phenyl]indolin-2-one, (0.122 g). MS: 372.18(M);371.18(M−1).

### Example 8

Preparation of 6-[4-((1S)-1-methyl-2-{[methylethyl)sulfonyl]amino}ethyl)phenyl}indolin-2-one.

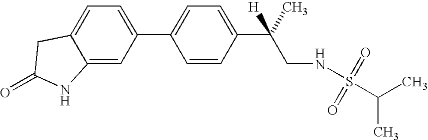

[(2S)-2-(4-iodophenyl)propyl][(methylethyl)sulfonyl]amine (0.330 g, 0.899 mmol), bis(pinacolate) diboron (0.250 g, 0.984mmol) PdCl₂(dppf).CH₂Cl₂ (0.024 g, 0.029 mmol) and KOAc (0.0.338 g, 3.44 mmol) were combined in dry DMF and heated at 80° C. under nitrogen for 24 h. The reaction mixture was allowed to cool to ambient temperature and 6-bromo-1,3-dihydro-indol-2-one.(0.209 g, 0.986 mmol), PdCl₂(dppf).CH₂Cl₂ (0.024 g, 0.029 mmol) and 2 M Na₂CO₃ (2.5 mL, 5.0 mmol) were added and the resulting mixture was stirred and heated under N₂ at 80° C. for 24 h. The reaction mixture was poured into EtOAc and filtered through Celite® and washed with brine. The organic layer was separated, dried (Na₂SO₄) is filtered, and the filtrate evaporated to give a brown oil. Chromatography on a 6 mm plate using the Chromatron® and eluting with a gradient EtOAc-hexane gave the title compound (0.103 g) as a white powder.

Analysis

Theory: C, 64.49; H, 6.49; N, 7.52

Found: C, 64.25; H, 6.21; N, 7.14

### Example 9

Preparation of 6-[4-((1R)-1-methyl-2-{[methylethyl)sulfonyl[amino}ethyl)phenyl-3-methyl-indolin-2-one.

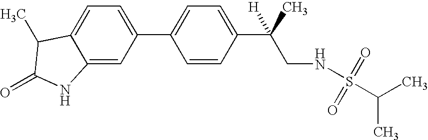

Preparation of 2-(4-Bromo-2-nitro-phenyl)propionic Acid.

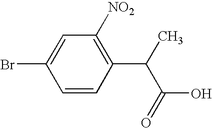

A mixture of 3-bromo-nitrotoluene (3.02 g, 12 mmol) and 2-chloro ethyl propionate (1.91 mL, 15.0 mmol) in anhydrous THF (15.0 mL) was added dropwise to a solution of 1.0 M KOtBu (30.0 mL) under N₂ at −40° C. over 10 min. The reaction turned a deep blue color. It was stirred at −45 to −35° C. for 1 h then allowed to warm to −10° C. It was then cooled again to −40° C. and quenched with 2 M HCl (20 mL). The mixture was diluted with EtOAc(175 mL). The organic layer was washed with brine, separated and dried (mgSO₄). Filtration followed by evaporation of the filtrate in vacuo gave a brown oil. The oil was placed onto silica gel in a 300 mL sintered glass funnel and eluted with EtOAc (1 L). Evaporation of the filtrate gave 1.45 g (40%) of the intermediate ester as a light brown oil. TLC (EtOAc-hexane 1:4), R_{f}=0.32. To a stirred solution of the ester in EtOH (10 mL) under N₂ was added a 5.26% solution of NaOH (28 mL) at ambient temperature. The resulting dark brown reaction mixture was stirred overnight ast ambient temperature then poured into H₂O (250 mL). The aqueous mixture was extracted with Et₂O (2×100 mL). The aqueous layer was separated and acidified to pH 1. The oily precipitate was extracted into EtOAc. The organic layer was separated washed with brine separated, filtered and dried (MgSO₄). Filtration and evaporation gave the intermediate title compound (0.83 g) as a waxy solid. MS(ES): 275.1(M+1).

6-Bromo-3-methyl-1,3-dihydro-indol-2-one.

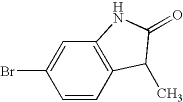

To a stirred solution of 2-(4-bromo-2-nitro-phenyl)propionic 0.800 g, 2.91 mmol) dissolved in 50% H₂SO₄ (6.2 mL)/EtOH (8.5 mL) was added Zn dust (0.76 g, 11.6 mmol) at 90° C. under nitrogen. The reaction was then treated in a manner analogous to the cyclization described above in example 6 to provide 0.61 g (93%) of 6-bromo-3-methyl-1,3-dihydro-indol-2-one.

Analysis

Theory: C, 47.82; H, 3.57; N, 6.20

Found: C, 47.80; H, 3.78; N, 5.83

Preparation of Final Title Compound.

Scheme I, step C: [(2R)-2-(4-Iodophenyl)propyl][(methylethyl)sulfonyl]amine (0.295 g, 0.804 mmol) bis(pinacolate) diboron (0.224 g,0.882 mmol) PdCl₂(dppf).CH₂Cl₂ (0.020 g, 0.024 mmol) and KOAc (0.0.276 g, 2.81 mmol) were combined and heated at 80° C. under nitrogen for 24 h. The mixture was allowed to cool to ambient temperature and 6-bromo-3-methyl-1,3-dihydro-1-indol-2-one (0.0.200 g, 0.885 mmol), PdCl₂(dppf).CH₂Cl₂ (0.020 g, 0.024 mmol)), 2M Na₂CO₃ (2.0 mL, 5.0 mmol) respectively were added. The resulting mixture was heated and stirred at 80° C. overnight and worked up in a manner analogous to the procedure described in example 3 to provide the final title compound, 6-[4-((1R)-1-methyl-2-{[(methylethyl)sulfonyl]amino}ethyl)phenyl]-3-methylindolin-2-one, (0.050 g). MS: 387(M+1).

### Example 10

Preparation of 7-[4-(1-methyl-2-{[methylethyl)sulfonyl]amino}-ethyl)phenyl]indolin-2-one

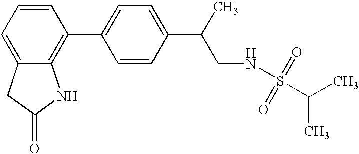

Preparation of 1-bromo-2-nitro-benzene Acetic Acid.

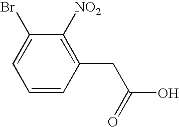

A mixture of 1-bromo-2-nitrotoluene (5.05 g, 25 mmol) and ethyl chloroacetate (3.68 g, 30 mmol) in anhydrous THF (30.0.0 mL) was added dropwise to a solution of 1.0 M KOtBu (60.0 mL 60 mmol) under N₂ at −40° C. over 12 min. The reaction turned a deep blue color. It was stirred at −45 to −35° C. for 30 min then allowed to warm to −10° C. It was then cooled again to −40° C. and quenched with 2 M HCl (40 mL). The mixture was diluted with EtOAc(150 mL). The organic layer was washed with brine, separated and dried (mgSO₄). Filtration followed by evaporation of the filtrate in vacuo gave a brown oil. The oil was flash chromatographed eluting with EtOAc-hexane 1:10 to give the crude ester. To a stirred solution of the crude ester in EtOH (50 mL) under N₂ was added 2M NaOH solution (100 mL) at ambient temperature. The resulting dark brown reaction mixture was stirred 16 h at ambient temperature . The resulting mixture was filtered and the filtrate was extracted with Et₂O (3×100 mL). The aqueous layer was acidified with HCl and the precipitated acid was extracted into EtOAc (100 mL). The organic layer was dried (Na₂SO₄) filtered and the filtrate evaporated to provide the intermediate title compound (2.04 g, 26%) as a dark brown crystalline solid.

Preparation of 7-Bromo-1,3-dihydro-indol-2-one.

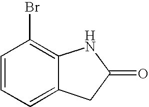

To a freshly prepared mixture of 1-bromo-2-nitro-benzene acetic acid (1.98 g, 7.61 mmol) in EtOH (20 mL) and 9M H₂SO₄ (20 mL) at 50° C. was added Zn dust( 2.00 g, 30.6 mmol) at ambient temperature. The reaction was stirred for 30 minutes at ambient temperature and then at 70° C. for 30 minutes. The reaction was cooled to ambient temperature and poured into H₂O (100 mL) and estracted with EtOAc (2×50 mL). The combined organic Layers were dried (Na₂SO₄), filtered and evaporated to give a crude brown residue. Chromatography on silica gel eluting with a gradient EtOAc-hexane system (1:10 to 1:1) gave the intermediate title compound (0.43 g, 27%) as a light brown solid.

Analysis

Theory: C, 45.31; H, 2.85; N, 6.60

Found: C, 45.01; H, 2.55; N, 6.42

Preparation of the Final Title Compound

2-[4-(4,4,5,5-tetramethyl-[1,3,2] dioxaborolan-2yl)-phenyl]propyl-2-propanesulfonamide (0.174 g, 0.47 mmol), 7-bromo-1,3-dihydro-indol-2-one (0.110 g, 0.52 mmol), PdCl₂(dppf).CH₂Cl₂(0.039 g, 0.048 mmol), and 2 M Na₂CO₃ (2.5 mL, 5.0 mmol) were combined in dry DMF (5.0 ml) and heated at 80° C. under nitrogen for 8 h. The reaction mixture turned a dark red violet after 1 h. It was then stirred at ambient temperature for 23 h. The reaction mixture was poured into H₂O (20 mL) and acidified with aq. HCl. A black sludge formed immediately. The mixture was decanted from the sludge and the sludge was extracted with Et₂O (4×20 mL). The combined organic layers were washed with H₂O 2×20 mL), dried (Na₂SO₄), filtered, and evaporated in vacuo. Chromatography of the resulting oil on the Chromatotron® using a 1 mm plate and eluting with EtOAc-hexane (1:1) gave the final title compound (0.068 g, 39%) as an orange red glass. MS(ES) 373 (M+1)

### Example 11

Preparation of 6-[4-(1-methyl-2-{[(methylethyl)sulfonyl]amino}ethyl)phenyl]-1,3,4-trihydroquinolin-2-one.

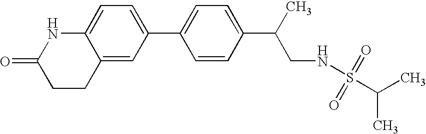

Scheme I, step C: 2-(4-Iodophenyl)propyl][(methylethyl)sulfonyl]amine (0.51 g, 1.40 mmol) and 6-(4,4,5,5-tetramethyl-[1,3,2]dioxaborolan-2-yl)-3,4-dihydro-1H-quinolin-2-one (0.42 g, 1.54 mmol, ) were combined with tetrakis(triphenylphosphine)palladium (0.05 g, 0.04 mmol) in 1,4-dioxane (6 mL). A 2 M solution of Na₂CO₃ (2.1 mL, 4.30 mmol) was added and the reaction mixture was heated at 60° C. under nitrogen for 8 hours then cooled to ambient temperature. The reaction mixture was dumped into diethyl-ether and washed with water and saturated NaCl, dried (MgSO₄), filtered, and concentrated to provide a brown oil which was purified by the Chromatotron® eluting with 30% ethyl acetate/hexanes to provide the final title compound, 6-[4-(1-methyl-2-{[(methylethyl)sulfonyl]amino}ethyl)phenyl]-1,3,4-trihydroquinolin-2-one, (0.13 g, 24%) as a light tan solid. MS(ES+): 387.2 (M+1); MS(ES−): 385.2 (M−1)

Analysis

Theory: C 65.25, H 6.78, N 7.25.

Found: C 65.14, H 6.18, N 6.55.

### Example 12

Preparation of 4-[4-(1-methyl-2-{[methylethyl)sulfonyl]amino}-ethyl)phenyl]indolin-2-one.

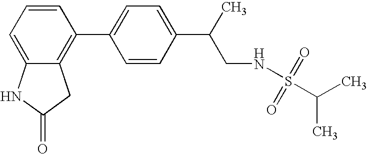

Preparation of 2-bromo-6-nitro-phenyl Acetic Acid

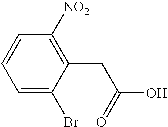

2-Bromo-6-nitro-phenyl acetic acid can be prepared according to the procedure of Magnus, et al. Tetrahedron Letters, 835-838 (2000). For example, pyrrolidine (1.10 mL, 0.013 mol) was added to a stirred solution of 2-bromo-6-nitrotoluene (3.24 g, 0.015 mol) and dimethylformamide dimethyl acetal in dry DMF (20 mL) at ambient temperature under N₂. The resulting mixture was heated and stirred for 3 h at 110° C. The dark brown reaction mixture was pioured into Et₂O—H₂O. The organic layer was extracted with H₂O, washed with brine, dried (MgSO₄), filtered and the filtrate evaporated. The brown residue was dissolved in a solution of THF (100 mL) at 0° C. To this solution was added 1.6 M HCl (50 mL) and the resulting mixture was stirred at 0-10° C. for 1 h then allowed to warm to ambient temperature and stirred for an hour and a half at ambient temperature. The mixture was poured into H₂O (400 mL) and extracted with Et₂O. The organic layer was dried (MgSO₄), filtered and evaporated to give a brown oil. The oil was dissolved in acetone (100 mL). To this solution at 0-10° C. was added a 10% solution of NaClO₂ (75 mL) and sulfamic acid(10.72 g, 0.104 mol) in H₂O (100 mL) followed by 10% Na₂HPO4 (75 mL). The resulting mixture was stirred at 0-10° C. for 1 h and poured into H₂O (300 mL). The aqueous mixture was extracted with Et₂O (200 mL). The organic layer was separated and extracted NaHCO₃ (2×100 mL). The aqueous layer was acidified with concentrated HCl and extracted with Et₂O (200 mL). The Et₂O layer was washed with brine, dried (MgSO₄), filtered and evaporated to give the intermediate title compound (0.78 g, 20%) as a pale yellow solid mp 159-161° C.(dec.).

Preparation of 4-Bromo-1,3-dihydro-indol-2-one

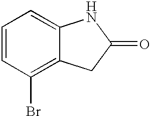

To a stirred solution of 2-bromo-6-nitro-phenyl acetic acid (0.66 g, 2.54 mmol) dissolved in 50% H₂SO₄ (6.0 mL)/EtOH (10.0 mL) was added Zn dust (0.66 g, 10.1 mmol) at 90° C. under N₂. The reaction mixture was then treated in a manner analogous to preparation 5 to provide the intermediate title compound (0.50 g, 93%). MS(ES) M+1 212, M+2 214.

Preparation of the Final Title Compound

2-[4-(4,4,5,5-tetramethyl-[1,3,2] dioxaborolan-2yl)-phenyl]propyl-2-propanesulfonamide (0.367, 1.0 mmol), 4-bromo-1,3-dihydro-indol-2-one (0.200 g, 0.94 mmol), PdCl₂(dppf).CH₂Cl₂ (0.033 g, 0.40 mmol), and 2 M Na₂CO₃ (3.5 mL, 7.0 mmol) were combined in dry DMF (25.0 mL) and heated at 80° C. under nitrogen for 8 h. The reaction mixture turned a dark bown color after 4 h. It was then stirred at ambient temperature for 72 h. The reaction mixture was poured into H₂O and extracted with EtOAc. The organic layer was separated and extracted several times with H₂O, washed with brine and dried (MgSO₄), filtered and evaporated in vacuo. Chromatography on the chromatron using a 4 mm plate and eluting with a gradient EtOAc/hexane 4:6 to 6:4 gave the final title compound, (0.086 g, 25%) as a brown solid.

MS(ES) 372 M+.

### Example 13

Preparation of 5-[4-(1-methyl-2-{[(methylethyl)sulfonyl]amino}ethyl)phenyl]-3-hydrobenzimidazol-2-one.

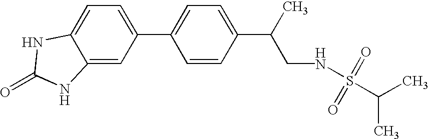

Preparation of 2-(4′-Amino-3′-nitro-biphenyl-4-yl)propyl-2-propane Sulfonamide.

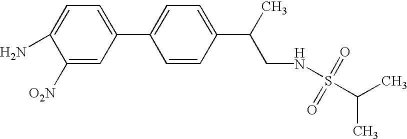

N-2-(4-Bromophenyl)propyl 2-propanesulfonamide (2.00 g, 6.24 mmol), bis(pinacolato)diboron (1.96 g, 7.70 mmol), PdCl₂(dppf).CH₂Cl₂ (0.211 g, 0.255 mmol) and KOAc (5.40 g, 55.0 mmol) were stirred and heated under nitrogen at 80° C. in dry DMF (50 mL) for 3 h. The reaction mixture was allowed to cool to ambient temperature and 4-bromo-2-nitro aniline (3.06 g, 1.4 mmol), PdCl₂(dppf).CH₂Cl₂ (0.211 g, 0.255 mmol), 2M Na₂CO₃ (17.62 mL, 35 mmol) were added. The reaction was then heated and stirred at 80° C. for 15 h. The reaction was worked up in a manner analogous to the procedure described in example 3, and chromatographed on the Chromatron® using a 4 mm plate and eluting with EtOAc/hexane (4:6) to provide the title intermediate compound, 2-(4′-amino-3′-nitro-biphenyl-4-yl)propyl-2-propane sulfonamide, (1.20 g, 51%) as a red solid, MS 376(M−1).

Analysis

Theory: C, 57.28;H, 6.14; N, 11.13

Found: C, 56.96;H, 5.92; N, 10.75

Preparation of 2-(3′,4′-Diamino-biphenyl-4-yl)propyl-2-propane Sulfonamide.

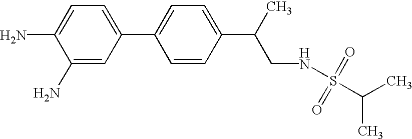

Stannous chloride dihydrate (0.57 g, 2.53 mmol) dissolved in EtOH (42 mL) was added to a suspension of 2-(4′-amino-3′-nitro-biphenyl-4-yl)propyl-2-propanesulonamide (0.191 g, 0.506 mmol) in 9M HCl (21.0 mL) at ambient temperature. The mixture warmed to 30° C. during addition. It was stirred for 1 h at ambient temperature then heated briefly at reflux. The resulting yellow solution was stirred for an additional 2 h at ambient temperature, evaporated to dryness in vacuo and the residue was dissolved in H₂O (100 mL). The aqueous solution was basified to pH 9.0 and the precipitated material extracted into EtOAc (150 mL). The EtOAc layer was washed with brine, dried (MgSO₄), and filtered. Evaporation of the solvent in vacuo provided the intermediate title compound, 2-(3′,4′-diamino-biphenyl-4-yl)propyl-2-propane sulfonamide, (0.088 g, 58%) which was used without further purification in the next step.

Preparation of Final Title Compound.

Triphosgene (0.47 g, 15.8 mmol) was added portionwise to a stirred suspension of 2-(3′,4′-diamino-biphenyl-4-yl)propyl-2-propane sulfonamide (0.088 g, 0.253 mmol) and 1N HCl (10.0 mL) in toluene(16 mL) at ambient temperature under nitrogen. The resulting mixture was stirred at ambient temperature over the weekend. It was basified with solid NaHCO₃ and filtered. The collected brown solid was washed with H₂O and toluene, and air dried. It was then recrystallized (CH₃OH—H₂O) to provide the final title compound, 5-[4-(1-methyl-2-{[(methylethyl)sulfonyl]amino}ethyl)phenyl]-3-hydrobenzimidazol-2-one, (0.052 g, 55%) as a fluffy brown solid. IR(KBr):1747 cm⁻¹

### Example 14

Preparation of 5[4-((1R)-1-methyl-2-{[(methylethyl)sulfonyl]amino}ethyl)phenyl]-3-hydrobenzimidazol-2-one

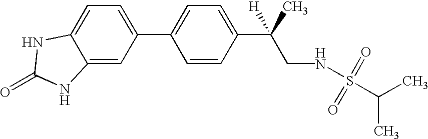

Preparation of 2-((2R)-2-methyl-(4′amino-3′-nitro-biphenyl-4yl)ethyl)-2-propanesulfonamide.

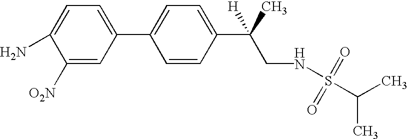

To a mixture of 4-bromo-2-nitroaniline (0.530 g, 2.4 mmol), 2M Na₂CO₃ (2.4 mL, 4.8 mmol), PdCl₂(dppf).CH₂Cl₂(0.180 g, 0.22 mmol), in DMF (10 mL) was added 2-((R)-2-methyl[4-(4,4,5,5-tetramethyl-[1,3,2] dioxaborolan-2yl)phenyl]ethyl)-2-propanesulfonamide (0.810 g, 2.2 mmol). The resulting mixture was heated and stirred at 80° C. under N₂ for 8 h then stirred at 25° C. for 15 h. The reaction was treated as described in example 12 to yield the intermediate title compound (0.720 g, 78%) as an orange glass.

Preparation of 2-((R)-2-methyl-(3′,4′-Diamino-biphenyl-4-yl)ethyl)l-2-propane sulfonamide.

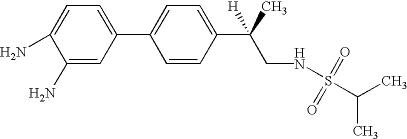

A mixture of 10% Pd/C (0.072 g) and the nitro-amino sulfonamide (0.720 g, 1.9 mmol) in MeOH (35 mL) was stirred unde 1 atm of H₂ for 17 h. The reaction mixture was filtered through Celite® and the deep violet filtrate was evaporated in vacuo providing the intermediate title compound (0.546 g) as a dark violet glass.

Preparation of 2-(2R)-[4-(4,4,5,5-tetramethyl-[1,3,2] dioxaborolan-2yl)phenyl]propyl-2-propanesulfonamide.

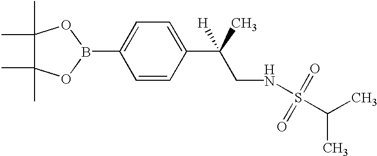

[(2R)-2-(4-Iodophenyl)propyl][(methylethyl)sulfonyl]amine (0.787 g, 2.14 mmol) bis(pinacolate) diboron (0.599 g,2.36 mmol) PdCl₂(dppf).CH₂Cl₂ (0.052 g, 0.064 mmol) and KOAc (0.630 g, 6.42 mmol) were combined in DMF(40 mL)and heated at 80° C. under nitrogen for 10 h. The resulting dark brown mixture was poured into a EtOAc and washed with H₂O and brine. The organic layer was dried (MgSO₄) and filtered. Evaporation of the filtrate followed by chromatography eluting with gave the intermediate title compound (1.0 g, 78%) as a white solid.

Analysis

Theory: C, 58.86; H, 8.23; N, 3.81.

Found: C, 58.84; H, 8.25; N, 3.96

Preparation of the Final Title Compound

To a suspension of 2 ((R)-2-methyl-(3′,4′-Diamino-biphenyl-4-yl)ethyl)l-2-propane sulfonamide (0.546 g, 1.57 mmol) and triphosgene in toluene (50 mL) was added 2 M aq. HCl (50 mL). The resulting mixture was stirred under N₂ for 18 h. The reaction mixture was basified with solid NaHCO₃ and filtered. The collected solid was washed with H₂O (3×20 mL), dissolved in boiling MeOH (50 mL) and filtered through a fritted glass funnel. The addition of H₂O followed by cooling at 4° C. for 4 h gave the final title compound (0.313 g, 51%) as a light mauve solid.

### Example 15

Preparation of 5-[4-(1-methyl-2-{[(methylethyl)sulfonyl]amino}ethyl)phenyl]-3-methylbenzimidazol-2-one.

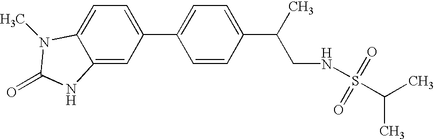

To a mixture of 6-bromo-1-ethoxycarbonyl-3-methyl(3H)-benzimidazolone (0.450 g, 1.52 mmol), 2M Na₂CO₃ (3.0 mL, 6.00 mmol) and PdCl₂(dppf).CH₂Cl₂ (0.120 g, 0.147 mmol) in DMF (10 mL) was added 2-[4-(4,4,5,5-tetramethyl-[1,3,2] dioxaborolan-2yl)-phenyl]propyl-2-propanesulfonamide (0.531 g, 1.45 mmol). The reaction mixture was stirred and heated under N₂ at 80° C. for 8 h and then stirred at 20° C. for 13 h. The reaction mixture was poured into a Et₂O (50 mL)-H₂O (50 mL) mixture and filtered. The collected semisolid was extracted with EtOAc (50 mL) and the EtOAc was evaporated to yield a tan solid (0.227 g). The solid was triturated with CH₂Cl₂ (5.0 mL), filtered, and then washed with CH₂Cl₂ (2×5.0 mL). After drying under high vacuum for 15 h the title compound (0.134 g, 24%) was isolated as a tan crystalline solid.

MS(ES) 388.13 (M+1).

The ability of compounds of formula I to potentiate glutamate receptor-mediated response may be determined using fluorescent calcium indicator dyes (Molecular Probes, Eugene, Oregon, Fluo-3) and by measuring glutamate-evoked efflux of calcium into GluR4 transfected HEK293 cells, as described in more detail below.

In one test, 96 well plates containing confluent monolayers of HEK 293 cells stably expressing human GluR4B (obtained as described in European Patent Application Publication Number EP-A1-583917) are prepared. The tissue culture medium in the wells is then discarded, and the wells are each washed once with 200 μl of buffer (glucose, 10 mM, sodium chloride, 138 mM, magnesium chloride, 1 mM, potassium chloride, 5 mM, calcium chloride, 5 mM, N-[2-hydroxyethyl]-piperazine-N-[2-ethanesulfonic acid], 10 mM, to pH 7.1 to 7.3). The plates are then incubated for 60 minutes in the dark with 20 μM Fluo3-AM dye (obtained from Molecular Probes Inc., Eugene, Oreg.) in buffer in each well. After the incubation, each well is washed once with 100 μl buffer, 200 μl of buffer is added and the plates are incubated for 30 minutes.

Solutions for use in the test are also prepared as follows. 30 μM, 10 μM, 3 μM and 1 μM dilutions of test compound are prepared using buffer from a 10 mM solution of test compound in DMSO. 100 μM cyclothiazide solution is prepared by adding 3 μl of 100 mM cyclothiazide to 3 ml of buffer. Control buffer solution is prepared by adding 1.5 μl DMSO to 498.5 μl of buffer.

Each test is then performed as follows. 200 μl of control buffer in each well is discarded and replaced with 45 μl of control buffer solution. A baseline fluorescent measurement is taken using a FLUOROSKAN II fluorimeter (Obtained from Labsystems, Needham Heights, Mass., USA, a Division of Life Sciences International Plc). The buffer is then removed and replaced with 45 μl of buffer and 45 μl of test compound in buffer in appropriate wells. A second fluorescent reading is taken after 5 minutes incubation. 15 μl of 400 μM glutamate solution is then added to each well (final glutamate concentration 100 μM), and a third reading is taken. The activities of test compounds and cyclothiazide solutions are determined by subtracting the second from the third reading (fluorescence due to addition of glutamate in the presence or absence of test compound or cyclothiazide) and are expressed relative to enhance fluorescence produced by 100 μM cyclothiazide.

In another test, HEK293 cells stably expressing human GluR4 (obtained as described in European Patent Application Publication No. EP-A1-0583917) are used in the electrophysiological characterization of AMPA receptor potentiators. The extracellular recording solution contains (in mM): 140 NaCl, 5 KCl, 10 HEPES, 1 MgCl₂, 2 CaCl₂, 10 glucose, pH=7.4 with NaOH, 295 mOsm kg-1. The intracellular recording solution contains (in mM): 140 CsCl, 1 MgCl₂, 10 HEPES, (N-[2-hydroxyethyl]piperazine-N1-[2-ethanesulfonic acid]) 10 EGTA (ethylene-bis(oxyethylene-nitrilo)tetraacetic acid), pH=7.2 with CsOH, 295 mOsm kg−1. With these solutions, recording pipettes have a resistance of 2-3 MΩ. Using the whole-cell voltage clamp technique (Hamill et al.(1981)Pflügers Arch., 391: 85-100), cells are voltage-clamped at −60 mV and control current responses to 1 mM glutamate are evoked. Responses to 1 mM glutamate are then determined in the presence of test compound. Compounds are deemed active in this test if, at a test concentration of 10 μM or less, they produce a greater than 10% increase in the value of the current evoked by 1 mM glutamate.

In order to determine the potency of test compounds, the concentration of the test compound, both in the bathing solution and co-applied with glutamate, is increased in half log units until the maximum effect was seen. Data collected in this manner are fit to the Hill equation, yielding an EC₅₀ value, indicative of the potency of the test compound. Reversibility of test compound activity is determined by assessing control glutamate 1 mM responses. Once the control responses to the glutamate challenge are re-established, the potentiation of these responses by 100 μM cyclothiazide is determined by its inclusion in both the bathing solution and the glutamate-containing solution. In this manner, the efficacy of the test compound relative to that of cyclothiazide can be determined.

According to another aspect, the present invention provides a pharmaceutical composition, which comprises a compound of formula I or a pharmaceutically acceptable salt thereof and a pharmaceutically acceptable diluent or carrier.

The pharmaceutical compositions are prepared by known procedures using well-known and readily available ingredients. In making the compositions of the present invention, the active ingredient will usually be mixed with a carrier, or diluted by a carrier, or enclosed within a carrier, and may be in the form of a capsule, sachet, paper, or other container. When the carrier serves as a diluent, it may be a solid, semi-solid, or liquid material which acts as a vehicle, excipient, or medium for the active ingredient. The compositions can be in the form of tablets, pills, powders, lozenges, sachets, cachets, elixirs, suspensions, emulsions, solutions, syrups, aerosols, ointments containing, for example, up to 10% by weight of active compound, soft and hard gelatin capsules, suppositories, sterile injectable solutions, and sterile packaged powders.

Some examples of suitable carriers, excipients, and diluents include lactose, dextrose, sucrose, sorbitol, mannitol, starches, gum, acacia, calcium phosphate, alginates, tragcanth, gelatin, calcium silicate, micro-crystalline cellulose, polyvinylpyrrolidone, cellulose, water syrup, methyl cellulose, methyl and propyl hydroxybenzoates, talc, magnesium stearate, and mineral oil. The formulations can additionally include lubricating agents, wetting agents, emulsifying and suspending agents, preserving agents, sweetening agents, or flavoring agents. Compositions of the invention may be formulated so as to provide quick, sustained, or delayed release of the active ingredient after administration to the patient by employing procedures well known in the art.

The compositions are preferably formulated in a unit dosage form, each dosage containing from about 1 mg to about 500 mg, more preferably about 5 mg to about 300 mg (for example 25 mg) of the active ingredient. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical carrier, diluent, or excipient. A more specific discussion of certain unit dosage forms are provided below followed by some typical formulations.

Capsules are prepared by mixing the compound with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like.

Natural and synthetic gums are also convenient, including acacia, alginates, methylcellulose, polyvinylpyrrolidine and the like. Polyethylene glycol, ethylcellulose and waxes can also serve as binders.

A lubricant is generally necessary in a tablet formulation to prevent the tablet and punches from sticking in the die. The lubricant is chosen from such slippery solids as talc, magnesium and calcium stearate, stearic acid and hydrogenated vegetable oils.

Tablet disintegrators are substances which swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethylcellulose, for example, may be used, as well as sodium lauryl sulfate.

Enteric formulations are often used to protect an active ingredient from the strongly acidic contents of the stomach. Such formulations are created by coating a solid dosage form with a film of a polymer which is insoluble in acidic environments, and soluble in basic environments. Exemplary films are cellulose acetate phthalate, polyvinyl acetate phthalate, hydroxypropyl methylcellulose phthalate and hydroxypropyl methylcellulose acetate succinate.

Tablets are often coated with sugar as a flavor and sealant, or with film-forming protecting agents to modify the dissolution properties of the tablet. The compounds may also be formulated as chewable tablets, by using large amounts of pleasant-tasting substances such as mannitol in the formulation, as is now well-established practice. Instantly dissolving tablet-like formulations are also now frequently used to assure that the patient consumes the dosage form, and to avoid the difficulty in swallowing solid objects that bothers some patients.

When it is desired to administer the combination as a suppository, the usual bases may be used. Cocoa butter is a traditional suppository base, which may be modified by addition of waxes to raise its melting point slightly. Water-miscible suppository bases comprising, particularly, polyethylene glycols of various molecular weights are in wide use, also.

Transdermal patches have become popular recently. Typically they comprise a resinous composition in which the drugs will dissolve, or partially dissolve, which is held in contact with the skin by a film which protects the composition. Many patents have appeared in the field recently. Other, more complicated patch compositions are also in use, particularly those having a membrane pierced with pores through which the compound of formula I is pumped by osmotic action.

The following formulation examples are illustrative only and are not intended to limit the scope of the invention in any way.

### Formulation I

Hard gelatin capsules are prepared using the following ingredients:

| | Quantify (mg/capsule) |
|---|---|
| Active Ingredient | 250 |
| Starch, dried | 200 |
| Magnesium Stearate | 10 |
| Total | 460 |

The above ingredients are mixed and filled into hard gelatin capsules in 460 mg quantities.

### Formulation 2

Tablets each containing 60 mg of active ingredient are made as follows:

| | Quantity (mg/tablet) |
|---|---|
| Active Ingredient | 60 |
| Starch | 45 |
| Microcrystalline Cellulose | 35 |
| Polyvinylpyrrolidone | 4 |
| Sodium Carboxymethyl Starch | 4.5 |
| Magnesium Stearate | 0.5 |
| Talc | 1 |
| Total | 150 |

As used herein the term “active ingredient” refers to a compound of formula I. The active ingredient, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50° C. and passed through a No. 18 mesh U.S. sieve. The sodium carboxymethyl starch, magnesium stearate, and talc, previously passed through a No. 60 mesh U.S. sieve, are then added to the granules which, after mixing, are compressed on a tablet machine to yield tablets each weighing 150 mg.

As used herein the term “patient” refers to a mammal, such as a mouse, guinea pig, rat, dog, horse, or human. It is understood that the preferred patient is a human.

As used herein, the terms “treating” or “to treat” each mean to alleviate symptoms, eliminate the causation either on a temporary or permanent basis, or to prevent or slow the appearance of symptoms of the named disorder. As such, the methods of this invention encompass both therapeutic and prophylactic administration.

As used herein, the term “effective amount” refers to the amount of a compound of formula I which is effective, upon single or multiple dose administration to a patient, in treating the patient suffering from the named disorder.

An effective amount can be readily determined by the attending diagnostician, as one skilled in the art, by the use of known techniques and by observing results obtained under analogous circumstances. In determining the effective amount or dose, a number of factors are considered by the attending diagnostician, including, but not limited to: the species of mammal; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The compounds can be administered by a variety of routes including oral, rectal, transdermal, subcutaneous, intravenous, intramuscular, bucal or intranasal routes. Alternatively, the compound may be administered by continuous infusion. A typical daily dose will contain from about 0.01 mg/kg to about 100 mg/kg of the active compound of this invention. Preferably, daily doses will be about 0.05 mg/kg to about 50 mg/kg, more preferably from about 0.1 mg/kg to about 25 mg/kg.

While all of the compounds of this invention are useful for potentiating glutamate receptor function in a patient, certain groups are preferred as follows:

With respect to R¹, compounds of formula I wherein R¹ is methyl, ethyl, isopropyl or N(CH₃)₂ are preferred with isopropyl being most preferred.

With respect to R², compounds of formula I wherein R² is hydrogen, methyl or ethyl are preferred, with hydrogen or methyl being most preferred.

With respect to R³, compound of formula I wherein R³ is hydrogen, methyl or ethyl are preferred, with hydrogen or methyl being most preferred.

In addition, when R² is methyl, it is most preferred that R³ is hydrogen, and when R² is hydrogen, it is most preferred that R³ is methyl.

With respect to R^{4a} and R^{4b}, compounds of formula I wherein R^{4a} and R^{4b} are each independently hydrogen, methyl, ethyl, methoxy, ethoxy, Br, Cl or F are preferred, with hydrogen, methyl, methoxy and F being most preferred.

With respect to R⁵, compounds of formula I wherein R⁵ is hydrogen or methyl are preferred.

With respect to R⁶, compounds of formula I wherein R⁶ is hydrogen or methyl are preferred.

With respect to R⁷ and R⁸, compounds of formula I wherein R⁷ and R⁸ are each independently hydrogen, methyl, or ethyl are preferred, with methyl being most preferred.

With respect to R⁹, compounds of formula I wherein R⁹ is hydrogen, methyl, or ethyl are preferred, with methyl being most preferred.

With respect to R¹⁰ and R¹¹, compounds of formula I wherein R¹⁰ and R¹¹ are each independently hydrogen, methyl, or ethyl are preferred, with hydrogen and methyl being most preferred.

With respect to Y and Z, compounds of formula I wherein Y is N when Z is O, Y is CH₂ when Z is O and Y is CH₂CH₂ when Z is O are preferred.

We claim:

1. A compound of the formula: 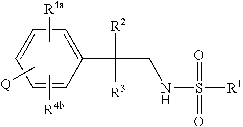
wherein
R¹ represents (1-6C)alkyl, (2-6C)alkenyl, or NR⁷R⁸;
R² and R³ each independently represent hydrogen, (1-4C)alkyl, or —OR⁹;
R^{4a} and R^{4b} each independently represent hydrogen, (1-4C) alkyl, (1-4C)alkoxy, I, Br, Cl, or F; and
Q is selected from the following: 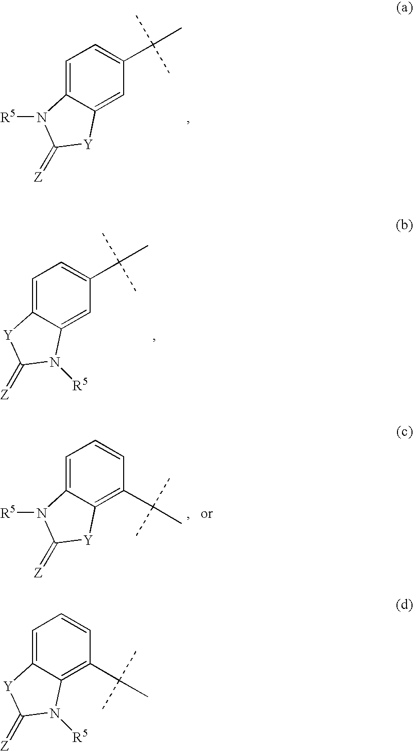
wherein
R⁵ represents hydrogen or (1-6C)alkyl;
Y represents CH₂CH₂, CR¹⁰R¹¹, NR⁶, S, or O;
Z represents O, S, or NH;
R⁶ represents hydrogen or (1-6C)alkyl;
R⁷ and R⁸ each independently represent hydrogen or (1-4C)alkyl;
R⁹ represents hydrogen or (1-4C)alkyl; and
R¹⁰ and R¹¹ each independently represent hydrogen or (1-4C)alkyl;
or a pharmaceutically acceptable salt thereof.

2. A compound according to claim 1 wherein R¹ is (1-4C)alkyl.

3. A compound according to claim 2 wherein R¹ is isopropyl.

4. A compound according to claim 3 wherein R² is (1-4C)alkyl and R³ is hydrogen.

5. A compound according to claim 4 wherein R² is methyl and R⁵ is hydrogen.

6. A compound according to claim 5 wherein R^{4a} and R^{4b} are each independently hydrogen, F, methyl, or methoxy.

7. A compound according to claim 6 wherein Z is O.

8. A compound according to claim 7 wherein Y is NR⁶.

9. A compound according to claim 8 wherein R⁶ is hydrogen.

10. A compound according to claim 7 wherein Y is CR¹⁰R¹¹.

11. A compound selected from the group consisting of:
6-[4-(1-methyl-2-{[(methylethyl)sulfonyl]amino}ethyl)phenyl]-3-hydrobenzothiazol-2-one;
6-[4-(1-methyl-2-{[(methylethyl)sulfonyl]amino}ethyl)phenyl]-3-hydrobenzoxazol-2-one;
5-[4-(1-methyl-2-([(methylethyl)sulfonyl]amino}ethyl)phenyl]indolin-2-one;
5-[4-((1R)-1-methyl-2-{[(methylethyl)sulfonyl]amino}ethyl)phenyl]indolin-2-one;
5-[4-((1S)-1-methyl-2-{[(methylethyl)sulfonyl]amino}ethyl)phenyl]indolin-2-one;
6-[4-(1-methyl-2-{[(methylethyl)sulfonyl]amino}ethyl)phenyl]indolin-2-one;
6-[4-((1R)-1-methyl-2-{[(methylethyl)sulfonyl]amino}ethyl)phenyl]indolin-2-one;
6-[4-(1-methyl-2-{[(methylethyl)sulfonyl]amino}ethyl)phenyl]-1,3,4-trihydroquinolin-2-one; and
5-[4-(1-methyl-2-{[(methylethyl)sulfonyl]amino}ethyl)phenyl]-3-hydrobenzimidazol-2-one; or a pharmaceutically acceptable salt thereof.

12. A pharmaceutical composition, which comprises a compound as claimed in any one of claims 1 to 11 and a pharmaceutically acceptable diluent or carrier.

13. A method of potentiating glutamate receptor function in a patient, which comprises administering to said patient an effective amount of a compound of formula: 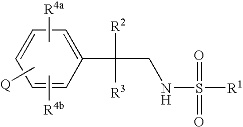
wherein
R¹ represents (1-6C)alkyl, (2-6C)alkenyl, or NR⁷R⁸;
R² and R³ each independently represent hydrogen, (1-4C)alkyl, or —OR⁹;
R^{4a} and R^{4b} each independently represent hydrogen, (1-4C) alkyl, (1-4C)alkoxy, I, Br, Cl, or F; and
Q is selected from the following: 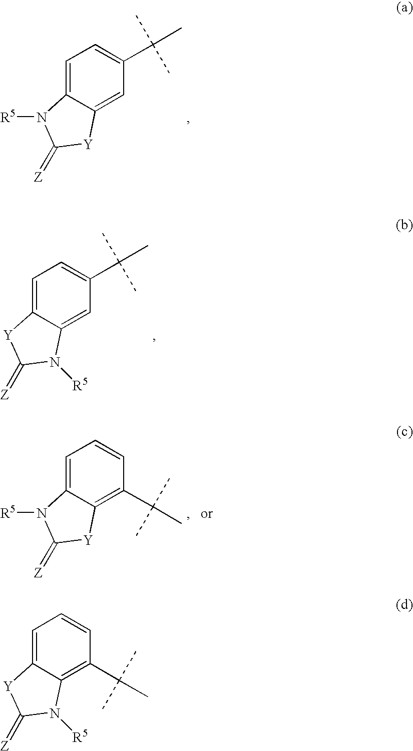
wherein
R⁵ represents hydrogen or (1-6C)alkyl;
Y represents CH₂CH₂, CR¹⁰R¹¹, NR⁶, S, or O;
Z represents O, S, or NH;
R⁶ represents hydrogen or (1-6C)alkyl;
R⁷ and R⁸ each independently represent hydrogen or (1-4C)alkyl;
R⁹ represents hydrogen or (1-4C)alkyl; and
R¹⁰ and R¹¹ each independently represent hydrogen or (1-4C)alkyl;
or a pharmaceutically acceptable salt thereof.

14. A method of treating a cognitive disorder; Alzheimer's disease, a neuro-degenerative disorder; age-related dementia; age-induced memory impairment; movement disorder; reversal of a drug-induced state; depression; attention deficit disorder; attention deficit hyperactivity disorder; psychosis; cognitive deficits associated with psychosis; drug-induced psychosis, or stroke in a patient, which comprises administering to a patient an effective amount of a compound of formula: 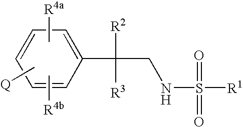
wherein
R¹ represents (1-6C)alkyl, (2-6C)alkenyl, or NR⁷R⁸;
R² and R³ each independently represent hydrogen, (1-4C)alkyl, or —OR⁹;
R^{4a} and R^{4b} each independently represent hydrogen, (1-4C) alkyl, (1-4C)alkoxy, I, Br, Cl, or F; and
Q is selected from the following: 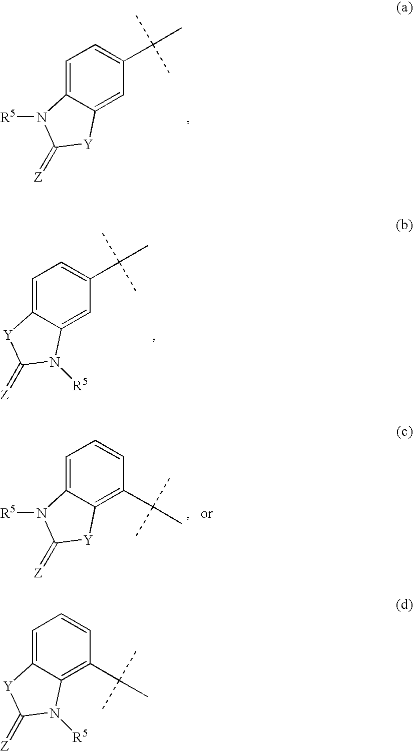
wherein
R⁵ represents hydrogen or (1-6C)alkyl;
Y represents CH₂CH₂, CR¹⁰R¹¹, NR⁶, S, or O;
Z represents O, S, or NH;
R⁶ represents hydrogen or (1-6C)alkyl;
R⁷ and R⁸ each independently represent hydrogen or (1-4C)alkyl;
R⁹ represents hydrogen or (1-4C)alkyl; and
R¹⁰ and R¹¹ each independently represent hydrogen or (1-4C)alkyl;
or a pharmaceutically acceptable salt thereof.

15. A compound according to any of claims 1 to 11, or a pharmaceutically acceptable salt thereof, for use as a pharmaceutical.

16. The use of a compound according to any of claims 1 to 11, or a pharmaceutically acceptable salt thereof, for the manufacture of a medicament for potentiating glutamate receptor function.

17. The use of a compound according to any of claims 1 to 11 for the manufacture of a medicament for treating a cognitive disorder; a neuro-degenerative disorder; age-related dementia; age-induced memory impairment; movement disorder; reversal of a drug-induced state; depression; attention deficit disorder; attention deficit hyperactivity disorder; psychosis; cognitive deficits associated with psychosis; drug-induced psychosis, or stroke.
